(19) United States
(12) Patent Application Publication
(10) Pub. No.: US 2022/0348652 A1
(43) Pub. Date: Nov. 3, 2022

(54) RECOMBINANT ANTIBODIES, PHARMACEUTICAL COMPOSITIONS COMPRISING THE SAME, AND USES THEREOF

(71) Applicant: AnTaimmu BioMed Co., Ltd., Hsinchu City (TW)

(72) Inventors: San-Tai SHEN, Hsinchu City (TW); Yueh-Liang TSOU, Hsinchu City (TW); Jen-Chieh LIN, Hsinchu City (TW); Chia-Chi LIU, Hsinchu City (TW)

(73) Assignee: AnTaimmu BioMed Co., Ltd., Hsinchu City (TW)

(21) Appl. No.: 17/726,537

(22) Filed: Apr. 22, 2022

Related U.S. Application Data
(60) Provisional application No. 63/178,543, filed on Apr. 23, 2021.

(51) Int. Cl. C07K 16/28

(52) U.S. Cl. CPC C07K 16/2803; C07K 2317/92; C07K 2317/622; C07K 2317/73; C07K 2317/76

(57) ABSTRACT

Disclosed herein are recombinant antibodies exhibiting binding affinity to CD47 polypeptide. According to some embodiments of the present disclosure, the recombinant antibodies are capable of blocking the interaction of CD47 and signal receptor protein-alpha (SIRPα). Accordingly, also disclosed herein are pharmaceutical compositions comprising the recombinant antibodies, and uses thereof in the treatment CD47-related diseases.

### CROSS-REFERENCE TO RELATED APPLICATIONS

This application relates to and claims the benefit of U.S. Provisional Application No. 63/178,543, filed Apr. 23, 2021; the content of the application is incorporated herein by reference in its entirety.

### BACKGROUND OF THE INVENTION

### 1. Field of the Invention

The present disclosure in general relates to the field of antibodies. More particularly, the present disclosure relates to recombinant antibodies and uses thereof in the treatment of CD47-associated diseases, e.g., cancers.

### 2. Description of Related Art

CD47 (cluster of differentiation 47), also known as integrin-associated protein (IAP), is a transmembrane protein involving in various cellular processes, including apoptosis, proliferation, adhesion, migration and angiogenesis, as well as immune responses. As an essential component of innate immune system, CD47 inhibits cellular phagocytosis via binding to signal receptor protein-alpha (SIRPα), a membrane protein expressed on phagocytic cells (such as macrophages, neutrophils and dendritic cells). Further, CD47 also exhibits an inhibitory effect on adaptive immune via binding to thrombospondin-1 (TSP1) that then suppresses T cell activation. Through interacting with the SIRPα and TSP-1 receptors, CD47 plays an important role in regulating immune evasion. Indeed, CD47 is overexpressed in various types of cancers, including solid cancers (e.g., gastric, colorectal, ovarian and breast cancers) and hematological cancers (e.g., non-Hodgkin's lymphoma and myeloid leukemia), that enables cancer cells to evade immune surveillance. In many malignancies, the expression level of CD47 correlates with an aggressive phenotype and an overall poor clinical prognosis.

Since the CD47/SIRPα interaction negatively regulates phagocytic clearance of tumor cells or other immune surveillance, blockage of the interaction may provide a potential means to treat cancers. Different anti-CD47 antibodies are developed for treating cancers. However, these antibodies are still in clinical trial phase, and is known to lead to various adverse effects, such as red blood cell (RBC) toxicity, thrombocytopenia, anemia, nausea, diarrhea, chill, fever, headache and/or fatigue. Accordingly, there remains a need to develop a novel antibody that specifically binds to CD47 and blocks the signaling of CD47 and SIRPα.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a recombinant antibody or the fragment thereof. According to embodiments of the present disclosure, the recombinant antibody or the antibody fragment comprises a light chain variable (VL) domain and a heavy chain variable (VH) domain, in which the VL domain comprises a first light chain complementarity determining region (CDR-L1), a second light chain CDR (CDR-L2), and a third light chain CDR (CDR-L3); and the VH domain comprises a first heavy chain CDR (CDR-H1), a second heavy chain CDR (CDR-H2), and a third heavy chain CDR (CDR-H3).

According to some embodiments, the recombinant antibody is designated as “CD47-2”, in which the CDR-L1, CDR-L2, CDR-L3, CDR-H1, CDR-H2 and CDR-H3 respectively comprise the amino acid sequences of SEQ ID NOs: 1, 2, 3, 5, 6 and 7. In certain preferred examples, the VL and VH domains of the recombinant antibody CD47-2 respectively comprise the amino acid sequences of SEQ ID NOs: 4 and 8.

According to some embodiments, the recombinant antibody is designated as “CD47-3”, in which the CDR-L1, CDR-L2, CDR-L3, CDR-H1, CDR-H2 and CDR-H3 respectively comprise the amino acid sequences of SEQ ID NOs: 9, 10, 11, 13, 14 and 15. In certain preferred examples, the VL and VH domains of the recombinant antibody CD47-3 respectively comprise the amino acid sequences of SEQ ID NOs: 12 and 16.

According to some embodiments, the recombinant antibody is designated as “CD47-4”, in which the CDR-L1, CDR-L2, CDR-L3, CDR-H1, CDR-H2 and CDR-H3 respectively comprise the amino acid sequences of SEQ ID NOs: 17, 18, 19, 21, 22 and 23. In certain preferred examples, the VL and VH domains of the recombinant antibody CD47-4 respectively comprise the amino acid sequences of SEQ ID NOs: 20 and 24.

According to certain embodiments, the recombinant antibody is designated as “CD47-5”, in which the CDR-L1, CDR-L2, CDR-L3, CDR-H1, CDR-H2 and CDR-H3 respectively comprise the amino acid sequences of SEQ ID NOs: 25, 18, 26, 28, 29 and 30. In some preferred examples, the VL and VH domains of the recombinant antibody CD47-5 respectively comprise the amino acid sequences of SEQ ID NOs: 27 and 31.

According to certain embodiments, the recombinant antibody is designated as “CD47-6”, in which the CDR-L1, CDR-L2, CDR-L3, CDR-H1, CDR-H2 and CDR-H3 respectively comprise the amino acid sequences of SEQ ID NOs: 32, 33, 26, 35, 36 and 37. In some preferred examples, the VL and VH domains of the recombinant antibody CD47-6 respectively comprise the amino acid sequences of SEQ ID NOs: 34 and 38.

According to alternative embodiments, the recombinant antibody is designated as “CD47-7”, in which the CDR-L1, CDR-L2, CDR-L3, CDR-H1, CDR-H2 and CDR-H3 respectively comprise the amino acid sequences of SEQ ID NOs: 39, 40, 41, 43, 44 and 45. In some preferred examples, the VL and VH domains of the recombinant antibody CD47-7 respectively comprise the amino acid sequences of SEQ ID NOs: 42 and 46.

According to some embodiments, the recombinant antibody is designated as “CD47-8”, in which the CDR-L1, CDR-L2, CDR-L3, CDR-H1, CDR-H2 and CDR-H3 respectively comprise the amino acid sequences of SEQ ID NOs: 47, 2, 48, 50, 51 and 52. In certain preferred examples, the VL and VH domains of the recombinant antibody CD47-8 respectively comprise the amino acid sequences of SEQ ID NOs: 49 and 53.

According to some embodiments, the recombinant antibody is designated as “CD47-9”, in which the CDR-L1, CDR-L2, CDR-L3, CDR-H1, CDR-H2 and CDR-H3 respectively comprise the amino acid sequences of SEQ ID NOs: 9, 54, 55, 57, 58 and 59. In certain preferred examples, the VL and VH domains of the recombinant antibody CD47-9 respectively comprise the amino acid sequences of SEQ ID NOs: 56 and 60.

According to certain embodiments, the recombinant antibody is designated as “CD47-14”, in which the CDR-L1, CDR-L2, CDR-L3, CDR-H1, CDR-H2 and CDR-H3 respectively comprise the amino acid sequences of SEQ ID NOs: 61, 62, 63, 13, 65 and 66. In certain preferred examples, the VL and VH domains of the recombinant antibody CD47-14 respectively comprise the amino acid sequences of SEQ ID NOs: 64 and 67.

According to certain embodiments, the recombinant antibody is designated as “CD47-20”, in which the CDR-L1, CDR-L2, CDR-L3, CDR-H1, CDR-H2 and CDR-H3 respectively comprise the amino acid sequences of SEQ ID NOs: 25, 33, 68, 70, 71 and 72. In certain preferred examples, the VL and VH domains of the recombinant antibody CD47-20 respectively comprise the amino acid sequences of SEQ ID NOs: 69 and 73.

According to certain embodiments, the recombinant antibody is designated as “CD47-22”, in which the CDR-L1, CDR-L2, CDR-L3, CDR-H1, CDR-H2 and CDR-H3 respectively comprise the amino acid sequences of SEQ ID NOs: 74, 75, 76, 78, 79 and 80. In certain preferred examples, the VL and VH domains of the recombinant antibody CD47-22 respectively comprise the amino acid sequences of SEQ ID NOs: 77 and 81.

According to alternative embodiments, the recombinant antibody is designated as “CD47-26”, in which the CDR-L1, CDR-L2, CDR-L3, CDR-H1, CDR-H2 and CDR-H3 respectively comprise the amino acid sequences of SEQ ID NOs: 82, 83, 84, 86, 87 and 72. In some preferred examples, the VL and VH domains of the recombinant antibody CD47-26 respectively comprise the amino acid sequences of SEQ ID NOs: 85 and 88.

According to alternative embodiments, the recombinant antibody is designated as “CD47-27”, in which the CDR-L1, CDR-L2, CDR-L3, CDR-H1, CDR-H2 and CDR-H3 respectively comprise the amino acid sequences of SEQ ID NOs: 89, 90, 91, 93, 94 and 66. In some preferred examples, the VL and VH domains of the recombinant antibody CD47-27 respectively comprise the amino acid sequences of SEQ ID NOs: 92 and 95.

According to some embodiments, the recombinant antibody is designated as “CD47-28”, in which the CDR-L1, CDR-L2, CDR-L3, CDR-H1, CDR-H2 and CDR-H3 respectively comprise the amino acid sequences of SEQ ID NOs: 96, 97, 98, 35, 100 and 72. In certain preferred examples, the VL and VH domains of the recombinant antibody CD47-28 respectively comprise the amino acid sequences of SEQ ID NOs: 99 and 101.

According to some embodiments, the recombinant antibody is designated as “CD47-29”, in which the CDR-L1, CDR-L2, CDR-L3, CDR-H1, CDR-H2 and CDR-H3 respectively comprise the amino acid sequences of SEQ ID NOs: 102, 103, 68, 35, 105 and 106. In certain preferred examples, the VL and VH domains of the recombinant antibody CD47-29 respectively comprise the amino acid sequences of SEQ ID NOs: 104 and 107.

According to certain embodiments, the recombinant antibody is designated as “CD47-30”, in which the CDR-L1, CDR-L2, CDR-L3, CDR-H1, CDR-H2 and CDR-H3 respectively comprise the amino acid sequences of SEQ ID NOs: 108, 109, 110, 112, 113 and 114. In certain preferred examples, the VL and VH domains of the recombinant antibody CD47-30 respectively comprise the amino acid sequences of SEQ ID NOs: 111 and 115.

According to certain embodiments, the recombinant antibody is designated as “CD47-31”, in which the CDR-L1, CDR-L2, CDR-L3, CDR-H1, CDR-H2 and CDR-H3 respectively comprise the amino acid sequences of SEQ ID NOs: 102, 116, 63, 13, 118 and 72. In certain preferred examples, the VL and VH domains of the recombinant antibody CD47-31 respectively comprise the amino acid sequences of SEQ ID NOs: 117 and 119.

According to alternative embodiments, the recombinant antibody is designated as “CD47-32”, in which the CDR-L1, CDR-L2, CDR-L3, CDR-H1, CDR-H2 and CDR-H3 respectively comprise the amino acid sequences of SEQ ID NOs: 120, 54, 121, 123, 124 and 59. In some preferred examples, the VL and VH domains of the recombinant antibody CD47-32 respectively comprise the amino acid sequences of SEQ ID NOs: 122 and 125.

It is the second aspect of this disclosure to provide a use of the recombinant antibody according to any of the above-mentioned embodiments for manufacturing a pharmaceutical composition. The pharmaceutical composition comprises a recombinant antibody described above, and a pharmaceutically acceptable carrier.

According to some preferred embodiments, the recombinant antibody is recombinant antibody CD47-2, CD47-7, CD47-14, CD47-22, CD47-26 or CD47-30.

Another aspect of the present disclosure pertains to a method of treating a CD47-related disease in a subject. The present method comprises administering to the subject an effective amount of the recombinant antibody, antibody fragment or pharmaceutical composition according to any of the above-mentioned aspects and embodiments.

The CD47-related disease may be any diseases or conditions associated with and/or caused by CD47 overexpression, for example, CD47-expressing cancers (i.e., cancers having CD47 overexpressed thereon/therein as compared to normal tissues/cells, in which CD47 as expressed in low levels). Non-limiting examples of CD47-expressing cancers treatable with the present method include, head and neck cancer, breast cancer, small cell lung cancer (SCLC), non-small cell lung cancer (NSCLC), liver cancer, gastric cancer, pancreatic cancer, colorectal cancer, ovarian cancer, bladder cancer, cholangiocarcinoma, glioblastoma, osteosarcoma, leiomyosarcoma (LMS), non-Hodgkin's lymphoma, acute myeloid leukemia (AML), acute lymphoblastic leukemia (ALL), chronic myeloid leukemia (CIVIL), myelodysplastic syndrome (MDS), and multiple myeloma (MM).

The subject treatable by the present method is a mammal, for example, human, mouse, rat, guinea pig, hamster, monkey, swine, dog, cat, horse, sheep, goat, cow, and rabbit. Preferably, the subject is a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is the data of ELISA depicting the binding of specified anti-CD47 single-chain variable fragments (scFvs) to CD47 polypeptide according to some embodiments of the present disclosure. NC: negative control; the scFv exhibiting no binding affinity to CD47.

FIG. 2 is a histogram depicting the binding of specified anti-CD47 scFvs toward CHO-CD47 cells, an engineered cell line stably expressing exogenous human CD47, according to Example 1 of the present disclosure. NC: negative control; the scFv exhibiting no binding affinity to CD47.

FIG. 3 is a histogram depicting the blocking effect of specified anti-CD47 scFvs on human SIRPα binding to CHO-CD47 cells according to Example 1 of the present disclosure. NC: negative control; the scFv exhibiting no binding affinity to CD47.

FIG. 4 is the data depicting the cell binding affinities of specified anti-CD47 IgGs to CHO-CD47 cells according to Example 2 of the present disclosure. R23: an anti-CD47 antibody, serving as a positive control.

FIG. 5 is a histogram depicting the blocking effect of specified anti-CD47 IgGs on human SIRPα binding to CHO-CD47 cells according to Example 2 of the present disclosure. R23: an anti-CD47 antibody, serving as a positive control.

FIG. 6 is a line graph depicting the antibody-dependent cellular phagocytosis (ADCP) reporter assay of specified anti-CD47 IgGs according to Example 2 of the present disclosure. R23: an anti-CD47 antibody, serving as a positive control.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “antibody” is used in the broadest sense and specifically covers monoclonal antibodies (including full length monoclonal antibodies), polyclonal antibodies, multi-specific antibodies (e.g., bi-specific antibodies), humanized antibodies, chimeric antibodies, and antibody fragments so long as they exhibit the desired biological activity. The term “antibody fragment” or “the fragment of antibody” refers to a portion of a full-length antibody, generally the antigen binding or variable domains (i.e., VL and VH domains) of a full-length antibody. Examples of the antibody fragment include fragment antigen-binding (Fab), Fab′, F(ab′)2, single-chain variable fragment (scFv), diabody, linear antibody, single-chain antibody molecule, and multi-specific antibody formed from antibody fragments.

The term “recombinant antibody,” as used herein, refers to antibodies that are prepared, expressed, created, or isolated by recombinant means, such as antibodies expressed using a recombinant expression vector transfected into a host cell, antibodies isolated from a recombinant or artificial antibody library, antibodies isolated from an animal (e.g., a mouse) that is transgenic for human immunoglobulin genes, or antibodies prepared, expressed, created or isolated by any other means that involves splicing of human immunoglobulin gene sequences to other DNA sequences.

The “variable domain” of an antibody refers to the amino-terminal domains of heavy or light chain of the antibody. These domains are generally the most variable parts of an antibody and contain the antigen-binding sites. The term “variable” refers to the fact that certain portions of the variable regions differ extensively in sequence among antibodies and are used in the binding and specificity of each particular antibody for its particular antigen. However, the variability is not evenly distributed throughout the variable regions of antibodies. It is concentrated in three segments called complementarity determining regions (CDRs) or hypervariable regions both in the light-chain and the heavy-chain variable regions. The more highly conserved portions of variable regions are called the framework (FR). The variable regions of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

The term “complementarity determining region (CDR)” used herein refers to the hypervariable region of an antibody molecule that forms a surface complementary to the 3-dimensional surface of a bound antigen. Proceeding from N-terminus to C-terminus, each of the antibody heavy and light chains comprises three CDRs (i.e., CDR-1, CDR-2, and CDR-3). A HLA-DR antigen-binding site, therefore, includes a total of six CDRs that comprise three CDRs from the variable region of a heavy chain (i.e., CDR-H1, CDR-H2, and CDR-H3), and three CDRs from the variable region of a light chain (i.e., CDR-L1, CDR-L2, and CDR-L3). The amino acid residues of CDRs are in close contact with bound antigen, wherein the closest antigen contact is usually associated with CDR-H3.

As discussed herein, minor variations in the amino acid sequences of antibodies are contemplated as being encompassed by the presently disclosed and claimed inventive concept(s), providing that the variations in the amino acid sequence maintain at least 85% sequence identity, such as at least 85%, 86%, 87%, 88%, 89%, 90%, 91%, 92%, 93%, 94%, 95%, 96%, 97%, 98% and 99% sequence identity. Antibodies of the present disclosure may be modified specifically to alter a feature of the peptide unrelated to its physiological activity. For example, certain amino acids can be changed and/or deleted without affecting the physiological activity of the antibody in this study (i.e., its ability to treat CD47-related disease). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of antibodies can be readily prepared by those of ordinary skill in the art. Preferred amino- and carboxyl-termini of fragments or analogs occur near boundaries of functional domains. In one example, one amino acid residue (e.g., valine) of the present antibody is conservatively replaced (e.g., by leucine). In other examples, two amino acid residues of the present antibody are conservatively replaced by other suitable amino acid residues, for example, valine (V) and arginine (R) are replaced by the pair of amino acids that includes, but is not limited to, methionine (M) and lysine (K), lysine (K) and proline (P), tryptophan (W) and isoleucine (I), isoleucine (I) and proline (P), asparagine (N) and valine (V), and glutamine (G) and lysine (K).

“Percentage (%) sequence identity” is defined as the percentage of amino acid residues in a candidate sequence that are identical with the amino acid residues in the specific peptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100$$

where X is the number of amino acid residues scored as identical matches by the sequence alignment program BLAST in that program's alignment of A and B, and where Y is the total number of amino acid residues in A or B, whichever is shorter.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Alternatively, the effective amount can be expressed in the concentration of the active component (e.g., the present recombinant antibody or the fragment thereof), such as molar concentration, mass concentration, volume concentration, molality, mole fraction, mass fraction and mixing ratio. Persons having ordinary skills could calculate the human equivalent dose (HED) for the medicament (such as the present recombinant antibody or the fragment thereof) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

As used herein, the term “treat,” “treating” and “treatment” are interchangeable, and encompasses partially or completely preventing, ameliorating, mitigating and/or managing a symptom, a secondary disorder or a condition associated with CD47-related diseases, e.g., cancers or infectious diseases. The term “treating” as used herein refers to application or administration of one or more recombinant antibodies of the present disclosure to a subject, who has a symptom, a secondary disorder or a condition associated with CD47-related diseases, e.g., cancers or infectious diseases, with the purpose to partially or completely alleviate, ameliorate, relieve, delay onset of, inhibit progression of, reduce severity of, and/or reduce incidence of one or more symptoms, secondary disorders or features associated with CD47-related diseases, e.g., cancers or infectious diseases. Symptoms, secondary disorders, and/or conditions associated with cancers include, but are not limited to, fatigue, weight changes, swelling or lumps, pain, fever, night sweats, cough, bleeding, bruising, jaundice and neurological problems (such as headaches, seizures, vision changes, hearing changes or drooping of the face). Symptoms, secondary disorders, and/or conditions associated with infectious diseases include, but are not limited to, fatigue, chill, fever, shock, cough, bleeding, pain, diarrhea, dehydration, congestion and itch. Treatment may be administered to a subject who exhibits only early signs of such symptoms, disorder, and/or condition for the purpose of decreasing the risk of developing the symptoms, secondary disorders, and/or conditions associated with CD47-related diseases. Treatment is generally “effective” if one or more symptoms or clinical markers are reduced as that term is defined herein. Alternatively, a treatment is “effective” if the progression of a symptom, disorder or condition is reduced or halted.

The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “subject” refers to a mammal including the human species that is treatable with the recombinant antibodies, pharmaceutical compositions and/or methods of the present invention. The term “subject” is intended to refer to both the male and female gender unless one gender is specifically indicated.

### II. Description of the Invention

The present disclosure provides 18 recombinant antibodies specific to CD47, and uses thereof in treating CD47-related diseases, such as cancers and infectious diseases.

(II-1) Preparation of the Present Recombinant Antibodies

According to some embodiments of the present disclosure, the recombinant antibodies are produced by phage-displayed scFv libraries, a technique well-known by a person having ordinary skill in the art that presents scFvs on the surface of lysogenic filamentous bacteriophages (i.e., viruses infecting bacteria, such as M13, T4, or T7). In general, the gene encoding the displayed molecule (i.e., scFv) is inserted into phage gene and expressed in fusion with phage coat protein. The phage display technique allows the creation of libraries that contain a great number of phage particles, in which each one encodes and displays different scFvs (10⁶-10¹¹ different scFvs in a population of >10¹² phage particles). Then, scFvs specific to a target molecule could be identified by affinity selection (also known as “biopanning”). For biopanning, a display library is incubated with an immobilized target molecule (e.g., CD47 polypeptide), followed by extensive washing to remove unbound or weakly-bound phages. The interacting phages are usually eluted using acidic buffer (e.g., glycine-HCl solution, pH2.2), high salt buffer (e.g., 2M NaCl) or high phosphate ion buffer (e.g., 500 mM NaH₂PO₄) and are enriched by amplification in the bacterial host cells (e.g., Escherichia coli, E. coli). Three to five rounds of biopanning are usually performed in order to obtain desired scFvs that bind to the target molecule with high affinity. The primary structure of the selected scFvs can then be determined by sequencing the DNA of individual clones.

According to some embodiments of the present disclosure, 18 scFvs specific to CD47 are selected via the biopanning procedure. These scFvs are respectively designated as “scFv CD47-2”, “scFv CD47-3”, “scFv CD47-4”, “scFv CD47-5”, “scFv CD47-6”, “scFv CD47-7”, “scFv CD47-8”, “scFv CD47-9”, “scFv CD47-14”, “scFv CD47-20”, “scFv CD47-22”, “scFv CD47-26”, “scFv CD47-27”, “scFv CD47-28”, “scFv CD47-29”, “scFv CD47-30”, “scFv CD47-31” and “scFv CD47-32” in the present disclosure.

The thus-obtained scFv is useful in the preparation of a recombinant antibody, which structurally comprises a VL domain, a light chain constant (CL) domain, a VH domain and a heavy chain constant (CH) domain. To construct a recombinant antibody, the phagemid DNA corresponding to the scFv-expressing phage is first extracted by lysing the phage via conventional DNA extraction technique; for example, the phenol/chloroform assay, and detergent (e.g., sodium dodecyl sulfate, TWEEN° -20, NP-40, and TRITON® X-100)/acetic acid assay. Then, the extracted phagemid DNA serves as a template to respectively amplify nucleic acid sequences encoding the CDR-L1, CDR-L2, CDR-L3, CDR-H1, CDR-H2, and CDR-H3 by polymerase chain reaction (PCR) using specific primers, followed by inserting the amplified nucleic acid sequences into an expression vector containing nucleic acid sequences respectively encoding the constant regions of the light and heavy chains of an immunoglobulin. Depending on desired purposes, the immunoglobulin can be any of IgG, IgA, IgD, IgE, and IgM. In one preferred embodiment of the present disclosure, the immunoglobulin is IgG. The thus-obtained expression vector is transfected into a host cell so as to produce the present recombinant antibody.

As could be appreciated, the present recombinant antibodies may alternatively be produced by conventional immunization method (i.e., immunizing animals with specific peptides). In brief, a target polypeptide (i.e., CD47 polypeptide) is synthesized and administered to a host animal, such as a mouse, a rat or a rabbit, thereby stimulating the immunized animal to produce antibodies against the target polypeptide. The immunization may be carried out at intervals of several days to several weeks, preferably one week, for 2-10 times, until a desired antibody titer is reached. Blood samples are taken regularly, and sera isolated therefrom are analyzed by suitable methods, such as enzyme linked immunosorbent assay (ELISA), enzyme immunoassay (EIA), or radio immunoassay (RIA), so as to determine antibody titers. After the final immunization, antibody-producing cells are isolated from splenic cells and regional lymph nodes of the immunized animals, and immediately fused with immortal cells such as myeloma cells (e.g., mouse myeloma FO cell line or human myeloma Karpas 707H cell line). Hybridomas of interest are selected from the fused cells via using HAT medium (a selection medium containing hypoxanthine, aminopterin and thymidine; only cells fused with antibody-producing cells may survive therein). Once antibody-positive cells are identified, cells are then cultured in a HT medium (a medium containing hypoxanthine and thymidine that is suitable for post-selection rescue to overcome the effects of residual hypoxanthine), and subjected to cloning to obtain single cells. The monoclonal antibodies produced by the hybridomas may be isolated or prepared by any known method. For example, affinity column, gel filtration chromatography, ion exchange chromatography or the like.

Alternatively, the present recombinant antibodies may be produced by DNA cloning. DNA encoding the present recombinant antibodies may be easily isolated and sequenced by use of conventional procedures, such as using oligonucleotide probes that are capable of binding specifically to genes encoding the heavy and light chains of the recombinant antibodies. The hybridoma cells serve as a preferred source of such DNA. Once isolated, the DNA may be placed into expression vectors, which are then transfected into host cells such as E. coli cells, simian COS cells or Chinese hamster ovary (CHO) cells or myeloma cells that do not produce immunoglobulin proteins, to synthesize the desired antibodies in the recombinant host cells. The invention also relates to such vectors comprising a nucleic acid sequence encoding the present recombinant antibody.

(II-2) Recombinant Antibodies

According to some embodiments of the present disclosure, 18 scFvs are selected by the biopanning procedure, and accordingly, 18 recombinant antibodies are produced therefrom. Thus, also disclosed herein are 18 recombinant antibodies, respectively designated as “antibody CD47-2”, “antibody CD47-3”, “antibody CD47-4”, “antibody CD47-5”, “antibody CD47-6”, “antibody CD47-7”, “antibody CD47-8”, “antibody CD47-9”, “antibody CD47-14”, “antibody CD47-20”, “antibody CD47-22”, “antibody CD47-26”, “antibody CD47-27”, “antibody CD47-28”, “antibody CD47-29”, “antibody CD47-30”, “antibody CD47-31” and “antibody CD47-32” in the present disclosure. As described above, depending on desired purposes, the recombinant antibody of the present disclosure may be prepared in the form of IgG, IgA, IgD, IgE or IgM. In one preferred embodiment of the present disclosure, the recombinant antibody is prepared in the form of IgG, in which the IgG antibodies are respectively designated as “CD47-2 IgG”, “CD47-3 IgG”, “CD47-4 IgG”, “CD47-5 IgG”, “CD47-6 IgG”, “CD47-7 IgG”, “CD47-8 IgG”, “CD47-9 IgG”, “CD47-14 IgG”, “CD47-20 IgG”, “CD47-22 IgG”, “CD47-26 IgG”, “CD47-27 IgG”, “CD47-28 IgG”, “CD47-29 IgG”, “CD47-30 IgG”, “CD47-31 IgG” and “CD47-32 IgG”.

In structure, each recombinant antibody or its fragment (e.g., scFv) comprises a VL domain and a VH domain, in which the VL domain comprises CDR-L1, CDR-L2 and CDR-L3, and the VH domain comprises CDR-H1, CDR-H2 and CDR-H3.

According to some embodiments, the CDR-L1, CDR-L2 and CDR-L3 of antibody CD47-2 or its fragment (e.g., scFv CD47-2) respectively have the amino acid sequences of SEQ ID NOs: 1-3 (i.e., respectively having the amino acid sequences 100% identical to SEQ ID NOs: 1-3), and the CDR-H1, CDR-H2 and CDR-H3 of antibody CD47-2 or its fragment (e.g., scFv CD47-2) respectively have the amino acid sequences of SEQ ID NOs: 5-7 (i.e., respectively having the amino acid sequences 100% identical to SEQ ID NOs: 5-7). According to some embodiments, the VL domain of antibody CD47-2 or its fragment (e.g., scFv CD47-2) comprises an amino acid sequence at least 85% (e.g., 85%, 86%, 87%, 88%, 89%, 90%, 91%, 92%, 93%, 94%, 95%, 96%, 97%, 98%, 99% or 100%) identical to SEQ ID NO: 4; and the VH domain of antibody CD47-2 or its fragment (e.g., scFv CD47-2) comprises an amino acid sequence at least 85% (e.g., 85%, 86%, 87%, 88%, 89%, 90%, 91%, 92%, 93%, 94%, 95%, 96%, 97%, 98%, 99% or 100%) identical to SEQ ID NO: 8. As could be appreciated, the sequences (e.g., the framework sequences) of the VL and VH domains may vary (e.g., being substituted by conserved or non-conserved amino acid residues) without affecting the binding affinity and/or specificity of the present antibody/antibody fragment. Preferably, the sequence(s) of the VL and VH domains is/are conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue). According to some preferred embodiments, the VL and VH domains of antibody CD47-2 or scFv CD47-2 respectively comprise amino acid sequences at least 90% identical to SEQ ID NOs: 4 and 8. More preferably, the VL and VH domains of antibody CD47-2 or scFv CD47-2 respectively comprise the amino acid sequences at least 95% identical to SEQ ID NOs: 4 and 8. In one working example of the present disclosure, the VL domain of antibody CD47-2 or scFv CD47-2 has the amino acid sequence of SEQ ID NO: 4 (i.e., having an amino acid sequence 100% identical to SEQ ID NO: 4), and the VH domain of antibody CD47-2 or scFv CD47-2 has the amino acid sequence of SEQ ID NO: 8 (i.e., having an amino acid sequence 100% identical to SEQ ID NO: 8).

According to some embodiments, the CDR-L1, CDR-L2 and CDR-L3 of antibody CD47-3 or its fragment (e.g., scFv CD47-3) respectively have the amino acid sequences of SEQ ID NOs: 9-11, and the CDR-H1, CDR-H2 and CDR-H3 of antibody CD47-3 or its fragment (e.g., scFv CD47-3) respectively have the amino acid sequences of SEQ ID NOs: 13-15. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-3 or scFv CD47-3 respectively comprise amino acid sequences at least 85% identical to SEQ ID NOs: 12 and 16; preferably, at least 90% identical to SEQ ID NOs: 12 and 16; more preferably, at least 95% identical to SEQ ID NOs: 12 and 16. In one working example of the present disclosure, the VL domain of antibody CD47-3 or scFv CD47-3 has the amino acid sequence of SEQ ID NO: 12, and the VH domain of antibody CD47-3 or scFv CD47-3 has the amino acid sequence of SEQ ID NO: 16.

According to some embodiments, the CDR-L1, CDR-L2 and CDR-L3 of antibody CD47-4 or its fragment (e.g., scFv CD47-4) respectively have the amino acid sequences of SEQ ID NOs: 17-19, and the CDR-H1, CDR-H2 and CDR-H3 of antibody CD47-4 or its fragment (e.g., scFv CD47-4) respectively have the amino acid sequences of SEQ ID NOs: 21-23. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-4 or scFv CD47-4 respectively comprise amino acid sequences at least 85% identical to SEQ ID NOs: 20 and 24; preferably, at least 90% identical to SEQ ID NOs: 20 and 24; more preferably, at least 95% identical to SEQ ID NOs: 20 and 24. In one working example of the present disclosure, the VL domain of antibody CD47-4 or scFv CD47-4 has the amino acid sequence of SEQ ID NO: 20, and the VH domain of antibody CD47-4 or scFv CD47-4 has the amino acid sequence of SEQ ID NO: 24.

According to some embodiments, the CDR-L1, CDR-L2 and CDR-L3 of antibody CD47-5 or its fragment (e.g., scFv CD47-5) respectively have the amino acid sequences of SEQ ID NOs: 25, 18 and 26, and the CDR-H1, CDR-H2 and CDR-H3 of antibody CD47-5 or its fragment (e.g., scFv CD47-5) respectively have the amino acid sequences of SEQ ID NOs: 28, 29 and 30. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-5 or scFv CD47-5 respectively comprise amino acid sequences at least 85% identical to SEQ ID NOs: 27 and 31; preferably, at least 90% identical to SEQ ID NOs: 27 and 31; more preferably, at least 95% identical to SEQ ID NOs: 27 and 31. In one working example of the present disclosure, the VL domain of antibody CD47-5 or scFv CD47-5 has the amino acid sequence of SEQ ID NO: 27, and the VH domain of antibody CD47-5 or scFv CD47-5 has the amino acid sequence of SEQ ID NO: 31.

According to certain embodiments, the CDR-L1, CDR-L2 and CDR-L3 of antibody CD47-6 or its fragment (e.g., scFv CD47-6) respectively have the amino acid sequences of SEQ ID NOs: 32, 33 and 26, and the CDR-H1, CDR-H2 and CDR-H3 of antibody CD47-6 or its fragment (e.g., scFv CD47-6) respectively have the amino acid sequences of SEQ ID NOs: 35, 36 and 37. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-6 or scFv CD47-6 respectively comprise amino acid sequences at least 85% identical to SEQ ID NOs: 34 and 38; preferably, at least 90% identical to SEQ ID NOs: 34 and 38; more preferably, at least 95% identical to SEQ ID NOs: 34 and 38. In one working example of the present disclosure, the VL domain of antibody CD47-6 or scFv CD47-6 has the amino acid sequence of SEQ ID NO: 34, and the VH domain of antibody CD47-6 or scFv CD47-6 has the amino acid sequence of SEQ ID NO: 38.

According to certain embodiments, the CDR-L1, CDR-L2 and CDR-L3 of antibody CD47-7 or its fragment (e.g., scFv CD47-7) respectively have the amino acid sequences of SEQ ID NOs: 39, 40 and 41, and the CDR-H1, CDR-H2 and CDR-H3 of antibody CD47-7 or its fragment (e.g., scFv CD47-7) respectively have the amino acid sequences of SEQ ID NOs: 43, 44 and 45. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-7 or scFv CD47-7 respectively comprise amino acid sequences at least 85% identical to SEQ ID NOs: 42 and 46; preferably, at least 90% identical to SEQ ID NOs: 42 and 46; more preferably, at least 95% identical to SEQ ID NOs: 42 and 46. In one working example of the present disclosure, the VL domain of antibody CD47-7 or scFv CD47-7 has the amino acid sequence of SEQ ID NO: 42, and the VH domain of antibody CD47-7 or scFv CD47-7 has the amino acid sequence of SEQ ID NO: 46.

According to certain embodiments, the CDR-L1, CDR-L2 and CDR-L3 of antibody CD47-8 or its fragment (e.g., scFv CD47-8) respectively have the amino acid sequences of SEQ ID NOs: 47, 2 and 48, and the CDR-H1, CDR-H2 and CDR-H3 of antibody CD47-8 or its fragment (e.g., scFv CD47-8) respectively have the amino acid sequences of SEQ ID NOs: 50, 51 and 52. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-8 or scFv CD47-8 respectively comprise amino acid sequences at least 85% identical to SEQ ID NOs: 49 and 53; preferably, at least 90% identical to SEQ ID NOs: 49 and 53; more preferably, at least 95% identical to SEQ ID NOs: 49 and 53. In one working example of the present disclosure, the VL domain of antibody CD47-8 or scFv CD47-8 has the amino acid sequence of SEQ ID NO: 49, and the VH domain of antibody CD47-8 or scFv CD47-8 has the amino acid sequence of SEQ ID NO: 53.

According to certain embodiments, the CDR-L1, CDR-L2 and CDR-L3 of antibody CD47-9 or its fragment (e.g., scFv CD47-9) respectively have the amino acid sequences of SEQ ID NOs: 9, 54 and 55, and the CDR-H1, CDR-H2 and CDR-H3 of antibody CD47-9 or its fragment (e.g., scFv CD47-9) respectively have the amino acid sequences of SEQ ID NOs: 57, 58 and 59. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-9 or scFv CD47-9 respectively comprise amino acid sequences at least 85% identical to SEQ ID NOs: 56 and 60; preferably, at least 90% identical to SEQ ID NOs: 56 and 60; more preferably, at least 95% identical to SEQ ID NOs: 56 and 60. In one working example of the present disclosure, the VL domain of antibody CD47-9 or scFv CD47-9 has the amino acid sequence of SEQ ID NO: 56, and the VH domain of antibody CD47-9 or scFv CD47-9 has the amino acid sequence of SEQ ID NO: 60.

According to alternative embodiments, the CDR-L1, CDR-L2 and CDR-L3 of antibody CD47-14 or its fragment (e.g., scFv CD47-14) respectively have the amino acid sequences of SEQ ID NOs: 61, 62 and 63, and the CDR-H1, CDR-H2 and CDR-H3 of antibody CD47-14 or its fragment (e.g., scFv CD47-14) respectively have the amino acid sequences of SEQ ID NOs: 13, 65 and 66. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-14 or scFv CD47-14 respectively comprise amino acid sequences at least 85% identical to SEQ ID NOs: 64 and 67; preferably, at least 90% identical to SEQ ID NOs: 64 and 67; more preferably, at least 95% identical to SEQ ID NOs: 64 and 67. In one working example of the present disclosure, the VL domain of antibody CD47-14 or scFv CD47-14 has the amino acid sequence of SEQ ID NO: 64, and the VH domain of antibody CD47-14 or scFv CD47-14 has the amino acid sequence of SEQ ID NO: 67.

According to alternative embodiments, the CDR-L1, CDR-L2 and CDR-L3 of antibody CD47-20 or its fragment (e.g., scFv CD47-20) respectively have the amino acid sequences of SEQ ID NOs: 25, 33 and 68, and the CDR-H1, CDR-H2 and CDR-H3 of antibody CD47-20 or its fragment (e.g., scFv CD47-20) respectively have the amino acid sequences of SEQ ID NOs: 70, 71 and 72. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-20 or scFv CD47-20 respectively comprise amino acid sequences at least 85% identical to SEQ ID NOs: 69 and 73; preferably, at least 90% identical to SEQ ID NOs: 69 and 73; more preferably, at least 95% identical to SEQ ID NOs: 69 and 73. In one working example of the present disclosure, the VL domain of antibody CD47-20 or scFv CD47-20 has the amino acid sequence of SEQ ID NO: 69, and the VH domain of antibody CD47-20 or scFv CD47-20 has the amino acid sequence of SEQ ID NO: 73.

According to some embodiments, the CDR-L1, CDR-L2 and CDR-L3 of antibody CD47-22 or its fragment (e.g., scFv CD47-22) respectively have the amino acid sequences of SEQ ID NOs: 74, 75 and 76, and the CDR-H1, CDR-H2 and CDR-H3 of antibody CD47-22 or its fragment (e.g., scFv CD47-22) respectively have the amino acid sequences of SEQ ID NOs: 78, 79 and 80. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-22 or scFv CD47-22 respectively comprise amino acid sequences at least 85% identical to SEQ ID NOs: 77 and 81; preferably, at least 90% identical to SEQ ID NOs: 77 and 81; more preferably, at least 95% identical to SEQ ID NOs: 77 and 81. In one working example of the present disclosure, the VL domain of antibody CD47-22 or scFv CD47-22 has the amino acid sequence of SEQ ID NO: 77, and the VH domain of antibody CD47-22 or scFv CD47-22 has the amino acid sequence of SEQ ID NO: 81.

According to some embodiments, the CDR-L1, CDR-L2 and CDR-L3 of antibody CD47-26 or its fragment (e.g. scFv CD47-26) respectively have the amino acid sequences of SEQ ID NOs: 82, 83 and 84, and the CDR-H1, CDR-H2 and CDR-H3 of antibody CD47-26 or its fragment (e.g. scFv CD47-26) respectively have the amino acid sequences of SEQ ID NOs: 86, 87 and 72. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-26 or scFv CD47-26 respectively comprise amino acid sequences at least 85% identical to SEQ ID NOs: 85 and 88; preferably, at least 90% identical to SEQ ID NOs: 85 and 88; more preferably, at least 95% identical to SEQ ID NOs: 85 and 88. In one working example of the present disclosure, the VL domain of antibody CD47-26 or scFv CD47-26 has the amino acid sequence of SEQ ID NO: 85, and the VH domain of antibody CD47-26 or scFv CD47-26 has the amino acid sequence of SEQ ID NO: 88.

According to certain embodiments, the CDR-L1, CDR-L2 and CDR-L3 of antibody CD47-27 or its fragment (e.g. scFv CD47-27) respectively have the amino acid sequences of SEQ ID NOs: 89, 90 and 91, and the CDR-H1, CDR-H2 and CDR-H3 of antibody CD47-27 or its fragment (e.g. scFv CD47-27) respectively have the amino acid sequences of SEQ ID NOs: 93, 94 and 66. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-27 or scFv CD47-27 respectively comprise amino acid sequences at least 85% identical to SEQ ID NOs: 92 and 95; preferably, at least 90% identical to SEQ ID NOs: 92 and 95; more preferably, at least 95% identical to SEQ ID NOs: 92 and 95. In one working example of the present disclosure, the VL domain of antibody CD47-27 or scFv CD47-27 has the amino acid sequence of SEQ ID NO: 92, and the VH domain of antibody CD47-27 or scFv CD47-27 has the amino acid sequence of SEQ ID NO: 95.

According to certain embodiments, the CDR-L1, CDR-L2 and CDR-L3 of antibody CD47-28 or its fragment (e.g., scFv CD47-28) respectively have the amino acid sequences of SEQ ID NOs: 96, 97 and 98, and the CDR-H1, CDR-H2 and CDR-H3 of antibody CD47-28 or its fragment (e.g., scFv CD47-28) respectively have the amino acid sequences of SEQ ID NOs: 35, 100 and 72. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-28 or scFv CD47-28 respectively comprise amino acid sequences at least 85% identical to SEQ ID NOs: 99 and 101; preferably, at least 90% identical to SEQ ID NOs: 99 and 101; more preferably, at least 95% identical to SEQ ID NOs: 99 and 101. In one working example of the present disclosure, the VL domain of antibody CD47-28 or scFv CD47-28 has the amino acid sequence of SEQ ID NO: 99, and the VH domain of antibody CD47-28 or scFv CD47-28 has the amino acid sequence of SEQ ID NO: 101.

According to some embodiments, the CDR-L1, CDR-L2 and CDR-L3 of antibody CD47-29 or its fragment (e.g. scFv CD47-29) respectively have the amino acid sequences of SEQ ID NOs: 102, 103 and 68, and the CDR-H1, CDR-H2 and CDR-H3 of antibody CD47-29 or its fragment (e.g. scFv CD47-29) respectively have the amino acid sequences of SEQ ID NOs: 35, 105 and 106. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-29 or scFv CD47-29 respectively comprise amino acid sequences at least 85% identical to SEQ ID NOs: 104 and 107; preferably, at least 90% identical to SEQ ID NOs: 104 and 107; more preferably, at least 95% identical to SEQ ID NOs: 104 and 107. In one working example of the present disclosure, the VL domain of antibody CD47-29 or scFv CD47-29 has the amino acid sequence of SEQ ID NO: 104, and the VH domain of antibody CD47-29 or scFv CD47-29 has the amino acid sequence of SEQ ID NO: 107.

According to some embodiments, the CDR-L1, CDR-L2 and CDR-L3 of antibody CD47-30 or its fragment (e.g., scFv CD47-30) respectively have the amino acid sequences of SEQ ID NOs: 108, 109 and 110, and the CDR-H1, CDR-H2 and CDR-H3 of antibody CD47-30 or its fragment (e.g., scFv CD47-30) respectively have the amino acid sequences of SEQ ID NOs: 112, 113 and 114. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-30 or scFv CD47-30 respectively comprise amino acid sequences at least 85% identical to SEQ ID NOs: 111 and 115; preferably, at least 90% identical to SEQ ID NOs: 111 and 115; more preferably, at least 95% identical to SEQ ID NOs: 111 and 115. In one working example of the present disclosure, the VL domain of antibody CD47-30 or scFv CD47-30 has the amino acid sequence of SEQ ID NO: 111, and the VH domain of antibody CD47-30 or scFv CD47-30 has the amino acid sequence of SEQ ID NO: 115.

According to alternative embodiments, the CDR-L1, CDR-L2 and CDR-L3 of antibody CD47-31 or its fragment (e.g., scFv CD47-31) respectively have the amino acid sequences of SEQ ID NOs: 102, 116 and 63, and the CDR-H1, CDR-H2 and CDR-H3 of antibody CD47-31 or its fragment (e.g., scFv CD47-31) respectively have the amino acid sequences of SEQ ID NOs: 13, 118 and 72. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-31 or scFv CD47-31 respectively comprise amino acid sequences at least 85% identical to SEQ ID NOs: 117 and 119; preferably, at least 90% identical to SEQ ID NOs: 117 and 119; more preferably, at least 95% identical to SEQ ID NOs: 117 and 119. In one working example of the present disclosure, the VL domain of antibody CD47-31 or scFv CD47-31 has the amino acid sequence of SEQ ID NO: 117, and the VH domain of antibody CD47-31 or scFv CD47-31 has the amino acid sequence of SEQ ID NO: 119.

According to alternative embodiments, the CDR-L1, CDR-L2 and CDR-L3 of antibody CD47-32 or its fragment (e.g., scFv CD47-32) respectively have the amino acid sequences of SEQ ID NOs: 120, 54 and 121, and the CDR-H1, CDR-H2 and CDR-H3 of antibody CD47-32 or its fragment (e.g., scFv CD47-32) respectively have the amino acid sequences of SEQ ID NOs: 123, 124 and 59. As described above, the framework sequences of the VL and VH domains of an antibody may vary (e.g., being substituted by conserved or non-conserved amino acid residues) without affecting the binding affinity and/or specificity of the antibody. According to some embodiments, the VL and VH domains of antibody CD47-32 or scFv CD47-32 respectively comprise amino acid sequences at least 85% identical to SEQ ID NOs: 122 and 125; preferably, at least 90% identical to SEQ ID NOs: 122 and 125; more preferably, at least 95% identical to SEQ ID NOs: 122 and 125. In one working example of the present disclosure, the VL domain of antibody CD47-32 or scFv CD47-32 has the amino acid sequence of SEQ ID NO: 122, and the VH domain of antibody CD47-32 or scFv CD47-32 has the amino acid sequence of SEQ ID NO: 125.

Optionally, the VL and VH domains of the present antibody fragment (e.g., scFv CD47-2, CD47-3, CD47-4, CD47-5, CD47-6, CD47-7, CD47-8, CD47-9, CD47-14, CD47-20, CD47-22, CD47-26, CD47-27, CD47-28, CD47-29, CD47-30, CD47-31 or CD47-32) are linked by a linker, which, according to some embodiments of the present disclosure, may be a cleavable or a non-cleavable linker. Optionally, the linker may be a flexible linker or an inflexible linker. The linker should be a length sufficiently long to allow the VL and VH domains to be linked without steric hindrance from one another and sufficiently short to retain the intended activity of the scFv. The linker preferably is sufficiently hydrophilic to avoid or minimize instability of the scFv. The linker should be sufficiently stable in vivo (e.g., it is not cleaved by serum, enzymes, etc.) to permit the scFv to be operative in vivo. Preferably, the linker consists of amino acid residues of glutamine (Q), serine (S), glycine (G), glutamate (E), proline (P), histidine (H) and/or arginine (R). More preferably, the linker consists of serine (S), glycine (G) and/or proline (P). Depending on intended purposes, the linker may have 1-25 amino acid residues in length, for example, having 1, 2, 3, 4, 5, 6, 7, 8, 9, 10, 11, 12, 13, 14, 15, 16, 17, 18, 19, 20, 21, 22, 23, 24 or 25 amino acid residues. According to certain exemplary embodiments, the linker comprises 1 to 5 units of the sequence of “GGP”; for example, the linker may comprise the amino acid sequence of “GGP”, “GGPGGP” (SEQ ID NO: 128), “GGPGGPGGP” (SEQ ID NO: 129), “GGPGGPGGPGGP” (SEQ ID NO: 130), or “GGPGGPGGPGGPGGP” (SEQ ID NO: 131). Alternatively, the linker may comprise the amino acid sequence of 1 to 5 units of the sequence of “GGGGS” (SEQ ID NO: 132); for example, the linker may comprise the amino acid sequence of “GGGGS” (SEQ ID NO: 132), “GGGGSGGGGS” (SEQ ID NO: 133), “GGGGSGGGGSGGGGS” (SEQ ID NO: 134), “GGGGSGGGGSGGGGSGGGGS” (SEQ ID NO: 135), or “GGGGSGGGGSGGGGSGGGGSGGGGS” (SEQ ID NO: 136).

According to some exemplary embodiments, the light chain constant (CL) and heavy chain constant (CH) domains of the present recombinant antibody (i.e., antibody CD47-2, CD47-3, CD47-4, CD47-5, CD47-6, CD47-7, CD47-8, CD47-9, CD47-14, CD47-20, CD47-22, CD47-26, CD47-27, CD47-28, CD47-29, CD47-30, CD47-31 or CD47-32) respectively comprise the amino acid sequences at least 85% identical to SEQ ID NOs: 126 and 127; preferably, at least 90% identical to SEQ ID NOs: 126 and 127; more preferably, at least 95% identical to SEQ ID NOs: 126 and 127. In one specific example, the CL and CH domains of the present recombinant antibody respectively comprise the amino acid sequences 100% identical to SEQ ID NOs: 126 and 127.

According to some embodiments of the present disclosure, each of the present recombinant antibodies and antibody fragments exhibits a binding affinity and blocking activity toward CD47 polypeptide, and accordingly, is useful in detecting CD47 expression and/or treating CD47-related diseases.

The present antibody fragment or recombinant antibody can be used to produce a multi-specific antibody (e.g., bi-specific antibody, BsAb; an antibody comprising two antibody/antibody fragment that can simultaneously bind to two different types of antigen or two different epitopes on the same antigen), a chimeric antibody (i.e., antibody consisting of domains from different species), a humanized antibody (i.e., the replacement of non-human constant regions and V framework regions for human sequences, results in a less immunogenic product), an antibody-drug conjugate (ADC; a biopharmaceutical drugs combining an antibody with a therapeutic agent), or a chimeric antigen receptor T cell (CAR T cell; i.e., T cells modified by adding a gene for a man-made receptor so as to improve the binding affinity and specificity of the modified T cells to a target antigen). Accordingly, the multi-specific antibody, chimeric antibody, humanized antibody, ADC and CAR T cell are also contemplated within the scope of the present disclosure.

Based on the CDR, VL and VH sequences of the present scFv or recombinant antibody, a person having ordinary skill in the art may produce different types of antibody fragments, such as F(ab′)2 (an antibody fragment containing two antigen-binding regions joined at the hinge through disulfides), Fab′ (an antibody fragment containing a free one antigen-binding region), Fab (an antibody fragment consisting of one VL, one CL, one VH and one CH1 regions), Fv (an antibody fragment containing VL and VH domains, which are linked by non-covalent interaction), and diabody (a noncovalent dimer of scFv). The methods for producing antibody fragments are known by a skilled artisan; hence, detailed description are omitted for the sake of brevity.

(II-3) Pharmaceutical Compositions

Also disclosed herein are pharmaceutical compositions comprising the recombinant antibody or antibody fragment (e.g., scFv) in accordance with any aspects or embodiments of the present disclosure. The present pharmaceutical composition comprises a recombinant antibody or the fragment thereof, and optionally, a pharmaceutically acceptable carrier.

Generally, the recombinant antibody/antibody fragment of this invention is present at a level of about 0.1% to 99% by weight, based on the total weight of the pharmaceutical composition. In some embodiments, the recombinant antibody/antibody fragment of this invention is present at a level of at least 1% by weight, based on the total weight of the pharmaceutical composition. In certain embodiments, the recombinant antibody/antibody fragment is present at a level of at least 5% by weight, based on the total weight of the pharmaceutical composition. In still other embodiments, the recombinant antibody/antibody fragment is present at a level of at least 10% by weight, based on the total weight of the pharmaceutical composition. In still yet other embodiments, the recombinant antibody/antibody fragment is present at a level of at least 25% by weight, based on the total weight of the pharmaceutical composition.

The present pharmaceutical composition may be formulated into solid, semi-solid, or liquid forms, such as tablets, capsules, powders, granules, ointments, solutions, suppositories, and injections. As such, administration of the present recombinant antibody/antibody fragment can be achieved in various ways, including oral, buccal, rectal, parental, intravenous, intraperitoneal, and etc. administration. When the present recombinant antibody/antibody fragment is formulated to be administered by intravenous, cutaneous or subcutaneous injection, the pharmaceutical composition will be formulated in the form of a pyrogen-free, parenterally acceptable aqueous solution. The preparation of such parenterally acceptable antibody solutions, having due regard to pH, isotonicity, stability, and the like, is within the skill in the art. A preferred pharmaceutical composition for intravenous, cutaneous, or subcutaneous injection should contain, in addition to the present recombinant antibody/antibody fragment, an isotonic vehicle such as sodium chloride injection, Ringer's injection, dextrose injection, dextrose and sodium chloride injection, lactated Ringer's injection, or other vehicle as known in the art. The pharmaceutical composition of the invention may also contain stabilizers, preservatives, buffers, antioxidants, or other additives known to those of skill in the art. The duration of intravenous therapy using the pharmaceutical composition of the invention will vary, depending on the severity of the disease being treated and the condition and potential idiosyncratic response of each individual subject. It is contemplated that the duration of each application of the present recombinant antibody/antibody fragment will be in the range of 12 to 24 hours of continuous intravenous administration. Ultimately the attending physician will decide on the appropriate duration of intravenous therapy.

In pharmaceutical dosage forms, the present recombinant antibody/antibody fragment may be administered alone or in combination with other known pharmaceutically active agent to treat diseases and conditions caused by CD47 overexpression (e.g., cancers or infectious diseases). One of skilled person in the art is familiar with the various dosage forms that are suitable for use in each route. It is to be noted that the most suitable route in any given case would depend on the nature or severity of the disease or condition being treated.

The pharmaceutical composition is prepared in accordance with acceptable pharmaceutical procedures, such as described in Remington's Pharmaceutical Sciences, 17^{th} edition, ed. Alfonoso R. Gennaro, Mack Publishing Company, Easton, Pa. (1985). Pharmaceutically acceptable carriers are those that are compatible with other ingredients in the formulation and biologically acceptable.

(II-4) Method for Treating CD47-Related Diseases

Another aspect of the present disclosure pertains to a method of treating a CD47-related disease in a subject. The method comprises administering to the subject an effective amount of the recombinant antibody, antibody fragment or pharmaceutical composition of the present disclosure.

The effective dose administered to the subject is from about 0.01 to 1,000 mg/Kg body weight of the subject, such as 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1,000 mg/Kg body weight of the subject; preferably, about 0.1 to 100 mg/Kg body weight of the subject. The dose can be administered in a single aliquot, or alternatively in more than one aliquot. The skilled artisan or clinical practitioner may adjust the dosage or regime in accordance with the physical condition of the patient or the severity of the diseases.

The CD47-related disease is a disease caused by and/or associated with CD47 overexpression; for example, cancers. It is known that CD47 is expressed at a low level in normal cells, while being overexpressed in various types of cancers so as to enable the cancers to evade immune surveillance. Accordingly, the present method is useful in treating cancers, especially CD47-expressing cancers. Non-limiting examples of CD47-expressing cancers include, head and neck cancer, breast cancer, small cell lung cancer (SCLC), non-small cell lung cancer (NSCLC), liver cancer, gastric cancer, pancreatic cancer, colorectal cancer, ovarian cancer, bladder cancer, cholangiocarcinoma, glioblastoma, osteosarcoma, leiomyosarcoma (LMS), non-Hodgkin's lymphoma, acute myeloid leukemia (AML), acute lymphoblastic leukemia (ALL), chronic myeloid leukemia (CML), myelodysplastic syndrome (MDS), and multiple myeloma (MM).

Alternatively, the present method may be useful in treating an infectious disease via blocking the immune-inhibitory activity of CD47 thereby enhancing immune responses against pathogens. The infectious disease may be caused by bacterium (e.g., Mycobacterium tuberculosis, Mycobacterium avium or Escherichia coli), virus (e.g., influenza virus, lymphocytic choriomeningitis virus (LCMV) or human immunodeficiency virus (HIV)), fungus (e.g., Candida albicans), parasite (e.g., Plasmodium falciparum or Plasmodium yoelii), or a combination thereof.

According to some preferred embodiments, the administration of the present recombinant antibody, antibody fragment or pharmaceutical composition does not induce hemagglutination of red blood cells or hemolysis in the subject.

Basically, the subject treatable by the present method is a mammal, for example, human, mouse, rat, guinea pig, hamster, monkey, swine, dog, cat, horse, sheep, goat, cow, and rabbit. Preferably, the subject is a human.

The present recombinant antibody, antibody fragment or pharmaceutical composition may be administered to the subject by a route selected from the group consisting of oral, enteral, nasal, topical, transmucosal, and parenteral administration, in which the parental administration is any of intramuscular, intravenous, or intraperitoneal injection.

As would be appreciated, the present method can be applied to the subject, alone or in combination with additional therapies that have some beneficial effects on the prevention or treatment of CD47-related diseases. Depending on the intended/therapeutic purpose, the present method can be applied to the subject before, during, or after the administration of the additional therapies.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

Materials and Methods

Preparations of CD47 Polypeptide

For the preparation of human CD47 polypeptide, DNA sequence encoding amino acids Met1-Pro139 of human CD47 (NM_001777.3) fused with a Fc region was constructed into mammalian expression vector. The constructed DNA was transfected into HEK293 cell. The CD47 polypeptide was then harvested in the supernatant and purified for the following biopanning procedure.

Library Construction and Phage Display Screening

A scFv library was constructed on phagemid vector. Before the first round of panning, the library was titrated and more than 10⁹ clones were collected. Purified human CD47 polypeptide were coated on a 96-well plate, and then 10¹¹-10¹² colony-forming unit (CFU) of polyethylene glycol (PEG) precipitated phages were add to each well of plate. The unbound phage was washed and the host E. coli was infected with bound phage. After two to three rounds of panning, single colony ELISA were assayed to confirm the binding of the selected phages.

Single Colony ELISA

Phages were infected into E. coli host cells followed by plating the infected E. coli cells on agar plate. Each colony was picked up and cultured in 2×YT (yeast extract tryptone) medium containing 100 μg/ml ampicillin with vigorous shacking at 37° C. After OD₆₀₀ of the culture was greater than 1, isopropyl β-d-1-thiogalactopyranoside (IPTG) was added to each culture and the final concentration of IPTG was 1 mM. The cultures were incubated at 37° C. overnight, and then centrifuged 4,000×g for 10 minutes. The scFvs were secreted and collected from the supernatant. The collected scFvs were added onto a 96-well plate coated with CD47 polypeptide followed by the analysis of ELISA so as to determining the binding affinity of the selected scFvs and CD47 polypeptide.

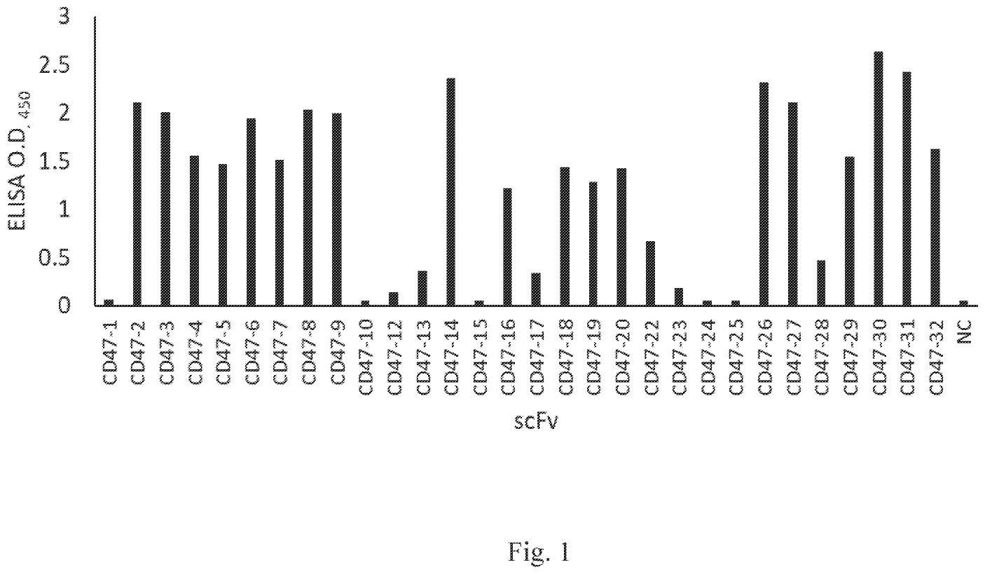

As the data depicted in FIG. 1, 30 scFvs were selected, in which 18 scFvs, including scFvs CD47-2, CD47-3, CD47-4, CD47-5, CD47-6, CD47-7, CD47-8, CD47-9, CD47-14, CD47-20, CD47-22, CD47-26, CD47-27, CD47-28, CD47-29, CD47-30, CD47-31 and CD47-32, exhibited a binding affinity to the CD47 protein. The CDR, VL and VH sequences of these anti-CD47 scFvs were summarized in Table 1 below.

TABLE 1
CDR, VL and VH sequences of specified scFvs

| | | | SEQ ID |
|---|---|---|---|
| Antibody | | Amino acid sequence | NO |
| CD47-2 | CDR-L1 | DVSSKVA | 1 |
| | CDR-L2 | YGTNRLYP | 2 |
| | CDR-L3 | QQSSNYPLT | 3 |
| | VL | DIQMTQSPSSLSASVGDRVTITCRASQDVSSKVAWYQQKP | 4 |
| | | GKAPKLLIYGTNRLYPGVPSRFSGSGSGTDFTLTISSLQPED | |
| | | FATYYCQQSSNYPLTFGQGTKVEIKR | |
| | CDR-H1 | FTISNSFIH | 5 |
| | CDR-H2 | YIWPWSGSTG | 6 |
| | CDR-H3 | ARGGFSMDH | 7 |
| | VH | EVQLVESGGGLVQPGGSLRLSCAASGFTISNSFIHWVRQAP | 8 |
| | | GKGLEWVAYIWPWSGSTGYADSVKGRFTISADTSKNTAY | |
| | | LQMNSLRAEDTAVYYCARGGFSMDHWGQGTLVTVSS | |
| CD47-3 | CDR-L1 | DVSNRVA | 9 |
| | CDR-L2 | SRANGLAS | 10 |
| | CDR-L3 | QQGSHFPIT | 11 |
| | VL | DIQMTQSPSSLSASVGDRVTITCRASQDVSNRVAWYQQKP | 12 |
| | | GKAPKLLISRANGLASGVPSRFSGSGSGTDFTLTISSLQPED | |
| | | FATYYCQQGSHFPITFGQGTKVEIKR | |
| | CDR-H1 | FTISNWWIH | 13 |
| | CDR-H2 | HIGPTVGKTY | 14 |
| | CDR-H3 | ARGGFAMDH | 15 |
| | VH | EVQLVESGGGLVQPGGSLRLSCAASGFTISNWWIHWVRQ | 16 |
| | | APGKGLEWVAHIGPTVGKTYYADSVKGRFTISADTSKNTA | |
| | | YLQMNSLRAEDTAVYYCARGGFAMDHWGQGTLVTVSS | |
| CD47-4 | CDR-L1 | SVDNGVA | 17 |
| | CDR-L2 | YSATRLYP | 18 |
| | CDR-L3 | QQSSTFPLT | 19 |
| | VL | DIQMTQSPSSLSASVGDRVTITCRASQSVDNGVAWYQQKP | 20 |
| | | GKAPKLLIYSATRLYPGVLSRFSGSGSGTDFTLTISSLQPED | |
| | | FATYYCQQSSTFPLTFGQGTKVEIKR | |
| | CDR-H1 | FTINDYWIH | 21 |
| | CDR-H2 | HIWPTYGGTH | 22 |
| | CDR-H3 | ARGGYSLDI | 23 |
| | VH | EVQLVESGGGLVQPGGSLRLSCAASGFTINDYWIHWVRQ | 24 |
| | | APGKGLEWVAHIWPTYGGTHYADSVKGRFTISADTSKNT | |
| | | AYLQMNSLRAEDAAVYYCARGGYSLDIWGQGTLVTVSS | |
| CD47-5 | CDR-L1 | DVHNKVA | 25 |
| | CDR-L2 | YSATRLYP | 18 |
| | CDR-L3 | QQHFNYPLT | 26 |
| | VL | DIQMTQSPSSLSASVGDRVTITCRASQDVHNKVAWYQQKP | 27 |
| | | GKAPKLLIYSATRLYPGVPSRFSGSGSGTDFTLTISSLQPED | |
| | | FATYYCQQHFNYPLTFGQGTKVEIKR | |
| | CDR-H1 | FTITNYWIH | 28 |
| | CDR-H2 | HIWPGVGDTD | 29 |
| | CDR-H3 | ARGGFAMDL | 30 |
| | VH | EVQLVESGGGLVQPGGSLRLSCAASGFTITNYWIHWVRQA | 31 |
| | | PGKGLEWVAHIWPGVGDTDYADSVKGRFTISADTSKNTA | |
| | | YLQMNSLRAEDTAVYYCARGGFAMDLWGQGTLVTVSS | |
| CD47-6 | CDR-L1 | DVSSRVA | 32 |
| | CDR-L2 | YYTSNLYS | 33 |
| | CDR-L3 | QQHFNYPLT | 26 |
| | VL | DIQMTQSPSSLSASVGDRVTITCRASQDVSSRVAWYQQKP | 34 |
| | | GKAPKLLIYYTSNLYSGVPSRFSGSGSGTDFTLTISSLQPED | |
| | | FATYYCQQHFNYPLTFGQGTKVEIKR | |
| | CDR-H1 | FTITNWWIH | 35 |
| | CDR-H2 | TISPYSGDTH | 36 |
| | CDR-H3 | ARGGYAMDH | 37 |
| | VH | EVQLVESGGGLVQPGGSLRLSCAASGFTITNWWIHWVRQ | 38 |
| | | APGKGLEWVATISPYSGDTHYADSVKGRFTISADTSKNTA | |
| | | YLQMNSLRAEDTAVYYCARGGYAMDHWGQGTLVTVSS | |
| CD47-7 | CDR-L1 | DVYTRVA | 39 |
| | CDR-L2 | YYTRNLYS | 40 |
| | CDR-L3 | QQAANYPLT | 41 |
| | VL | DIQMTQSPSSLSASVGDRVTITCRASQDVYTRVAWYQQKP | 42 |
| | | GKAPKLLIYYTRNLYSGVPSRFSGSGSGTDFTLTISSLQPED | |
| | | FATYYCQQAANYPLTFGQGTKVEIKR | |
| | CDR-H1 | TITNYYIH | 43 |
| | CDR-H2 | SIWPGYGNTG | 44 |
| | CDR-H3 | ARGGYSMDF | 45 |
| | VH | EVQLVESGGGLVQPGGSLRLSCAASGFTITNYYIHWVRQA | 46 |
| | | PGKGLEWVASIWPGYGNTGYADSVKGRFTISADTSKNTA | |
| | | YLQMNSLRAEDTAVYYCARGGYSMDFWGQGTLVTVSS | |
| CD47-8 | CDR-L1 | DVHYAVA | |
| | CDR-L2 | YGTNRLYP | 2 |
| | CDR-L3 | QQHSNFPLT | 48 |
| | VL | DIQMTQSPSSLSASVGDRVTITCRASQDVHYAVAWYQ | 49 |
| | | QKPGKAPKLLIYGTNRLYPGVPSRFSGSGSGTDFTLTISS | |
| | | LQPEDFATYYCQQHSNFPLTFGQGTKVEIKR | |
| | CDR-H1 | FTINNFYIH | 50 |
| | CDR-H2 | SIWPWSGETG | 51 |
| | CDR-H3 | ARGGFAMDF | 52 |
| | VH | EVQLVESGGGLVQPGGSLRLSCAASGFTINNFYIHWVR | 53 |
| | | QAPGKGLEWVASIWPWSGETGYADSVKGRFTIGADTS | |
| | | KNTAYLQMNSLRAEDTAVYYCARGGFAMDFWGQGTL | |
| | | VTVSS | |
| CD47-9 | CDR-L1 | DVSNRVA | 9 |
| | CDR-L2 | YGTNRLYT | 54 |
| | CDR-L3 | QQHFNYPIT | 55 |
| | VL | DIQMTQSPSSLSASVGDRVTITCRASQDVSNRVAWYQQ | 56 |
| | | KPGKAPKLLIYGTNRLYTGVPSRFSGSGSGTDFTLTISSL | |
| | | QPEDFATYYCQQHFNYPITFGQGTKVEIKR | |
| | CDR-H1 | FTINSWWIH | 57 |
| | CDR-H2 | KISPFNGDTH | 58 |
| | CDR-H3 | ARGGYAMDI | 59 |
| | VH | EVQLVESGGGLVQPGGSLRLSCAASGFTINSWWIHWV | 60 |
| | | RQAPGKGLEWVAKISPFNGDTHYADSVKGRFTISADTS | |
| | | KNTAYLQMNSLRAEDTAVYYCARGGYAMDIWGQGTL | |
| | | VTVSS | |
| CD47-14 | CDR-L1 | DVSTKVA | 61 |
| | CDR-L2 | YFTRNLYS | 62 |
| | CDR-L3 | QQHSNYPVT | 63 |
| | VL | DIQMTQSPSSLSASVGDRVTITCRASQDVSTKVAWYQQ | 64 |
| | | KPGKAPKLLIYFTRNLYSGVPSRFSGSGSGTDFTLTISSL | |
| | | QPEDFATYYCQQHSNYPVTFGQGTKVEIKR | |
| | CDR-H1 | FTISNWWIH | 13 |
| | CDR-H2 | GISPYSGKTD | 65 |
| | CDR-H3 | ARGGYAMDL | 66 |
| | VH | EVQLVESGGGLVQPGGSLRLSCAASGFTISNWWIHWV | 67 |
| | | RQAPGKGLEWVAGISPYSGKTDYADSVKGRFTISADTS | |
| | | KNTAYLQMNSLRAEDTAVYYCARGGYAMDLWGQGT | |
| | | LVTVSS | |
| CD47-20 | CDR-L1 | DVHNKVA | |
| | CDR-L2 | YYTSNLYS | 33 |
| | CDR-L3 | QQHFTYPLT | 68 |
| | | DIQMTQSPSSLSASVGDRVTITCRASQDVHNKVAWYQ | |
| | VL | QKPGKAPKLLIYYTSNLYSGVPSRFSGSGSGTDFTLTISS | 69 |
| | | LQPEDFATYYCQQHFTYPLTFGQGTKVEIKR | |
| | CDR-H1 | FTISNYYIH | 70 |
| | CDR-H2 | GIWPSGGSTS | 71 |
| | CDR-H3 | TRGGYAMDY | 72 |
| | | EVQLVESGGGLVQPGGSLRLSCAASGFTISNYYIHWVR | |
| | VH | QAPGKGLEWVAGIWPSGGSTSYADSVKGRFTISADTSK | 73 |
| | | NTAYLQMNSLRAEDTAVYYCTRGGYAMDYWGQGTL | |
| | | VTVSS | |
| CD47-22 | CDR-L1 | DVHSWVA | 74 |
| | CDR-L2 | SRATWLAS | 75 |
| | CDR-L3 | QQSSNFPLT | 76 |
| | VL | DIQMTQSPSSLSASVGDRVTITCRASQDVHSWVAWYQ | |
| | | QKPGKAPKLLISRATWLASGVPSRFSGSGSGTDFTLTIS | 77 |
| | | SLQPEDFATYYCQQSSNFPLTFGQGTKVEIKR | |
| | CDR-H1 | FTINDYGIH | 78 |
| | CDR-H2 | GIGPFWGSTF | 79 |
| | CDR-H3 | ARWMWGRWSAMDY | 80 |
| | VH | EVQLVESGGGLVQPGGSLRLSCAASGFTINDYGIHWVR | 81 |
| | | QAPGKGLEWVAGIGPFWGSTFYADSVKGRFTISADTSK | |
| | | NTAYLQMNSLRAEDTAVYYCARWMWGRWSAMDYW | |
| | | GQGTLVTVSS | |
| CD47-26 | CDR-L1 | DVSNYVA | 82 |
| | CDR-L2 | YGTSRLYP | 83 |
| | CDR-L3 | QQHSSYPLT | 84 |
| | VL | DIQMTQSPSSLSASVGDRVTITCRASQDVSNYVAWYQQ | |
| | | KPGKAPKLLIYGTSRLYPGVPSRFSGSGSGTDFTLTISSL | 85 |
| | | QPEDFATYYCQQHSSYPLTFGQGTKVEIKR | |
| | CDR-H1 | FTITNFYIH | 86 |
| | CDR-H2 | KITPYNGDTH | 87 |
| | CDR-H3 | TRGGYAMDY | 72 |
| | VH | EVQLVESGGGLVQPGGSLRLSCAASGFTITNFYIHWVR | 88 |
| | | QAPGKGLEWVAKITPYNGDTHYADSVKGRFTISADTS | |
| | | KNTAYLQMNSLRAEDTAVYYCTRGGYAMDYWGQGT | |
| | | LVTVSS | |
| CD47-27 | CDR-L1 | DVHNGVA | 89 |
| | CDR-L2 | FRTSWLLS | 90 |
| | CDR-L3 | QQGSSFPFT | 91 |
| | VL | DIQMTQSPSSLSASVGDRVTITCRASQDVHNGVAWYQ | 92 |
| | | QKPGKAPKLLIFRTSWLLSGVPSRFSGSGSGTDFTLTISS | |
| | | LQPEDFATYYCQQGSSFPFTFGQGTKVEIKR | |
| | CDR-H1 | FTINNYWIH | 93 |
| | CDR-H2 | RISPFDGGTY | 94 |
| | CDR-H3 | ARGGYAMDL | 66 |
| | VH | EVQLVESGGGLVQPGGSLRLSCAASGFTINNYWIHWV | 95 |
| | | RQAPGKGLEWVARISPFDGGTYYADSVKGRFTISADTS | |
| | | KNTAYLQMNSLRAEDTAVYYCARGGYAMDLWGQGT | |
| | | LVTVSS | |
| CD47-28 | CDR-L1 | DVHGKVA | 96 |
| | CDR-L2 | YYARNLYS | 97 |
| | CDR-L3 | QQYFNFPLT | 98 |
| | VL | DIQMTQSPSSLSASVGDRVTITCRASQDVHGKVAWYQ | 99 |
| | | QKPGKAPKLLIYYARNLYSGVPSRFSGSGSGTDFTLTIS | |
| | | SLQPEDFASYYCQQYFNFPLTFGQGTKVEIKR | |
| | CDR-H1 | FTITNWWIH | 35 |
| | CDR-H2 | EINPFGGNTY | 100 |
| | CDR-H3 | TRGGYAMDY | 72 |
| | VH | EVQLVESGGGLVQPGGSLRLSCAASGFTITNWWIHWV | 101 |
| | | RQAPGKGLEWVAEINPFGGNTYYADSVKGRFTISADTS | |
| | | KNTAYLQMNSLRAEDTAVYYCTRGGYAMDYWGQGT | |
| | | LVTVSS | |
| CD47-29 | CDR-L1 | DVHSKVA | 102 |
| | CDR-L2 | YSTNRLYP | 103 |
| | CDR-L3 | QQHFTYPLT | 68 |
| | VL | DIQMTQSPSSLSASVGDRVTITCRASQDVHSKVAWYQQ | 104 |
| | | KPGKAPKLLIYSTNRLYPGVPSRFSGSGSGTDFTLTISSL | |
| | | QPEDFATYYCQQHFTYPLTFGQGTKVEIKR | |
| | CDR-H1 | FTITNWWIH | 35 |
| | CDR-H2 | GITPYSGDTY | 105 |
| | CDR-H3 | ARGGYAIDN | 106 |
| | VH | EVQLVESGGGLVQPGGSLRLSCAASGFTITNWWIHWV | 107 |
| | | RQAPGKGLEWVAGITPYSGDTYYADSVKGRFTISADTS | |
| | | KNTAYLQMNSLRAEDTAVYYCARGGYAIDNWGQGTL | |
| | | VTVSS | |
| CD47-30 | CDR-L1 | DVSHYVA | |
| | CDR-L2 | YSTSRLYP | 109 |
| | CDR-L3 | QQHSSYPIT | 110 |
| | VL | DIQMTQSPSSLSASVGDRVTITCRASQDVSHYVAWYQQ | 111 |
| | | KPGKAPKLLIYSTSRLYPGVPSRFSGSGSGTDFTLTISSL | |
| | | QPEDFATYYCQQHSSYPITFGQGTKVEIKR | |
| | CDR-H1 | FTINNWWIH | 112 |
| | CDR-H2 | WIDPFNGDSD | 113 |
| | CDR-H3 | ARGGFALDH | 114 |
| | VH | EVQLVESGGGLVQPGGSLRLSCAASGFTINNWWIHWV | 115 |
| | | RQAPGKGLEWVAWIDPFNGDSDYADSVKGRFTISADT | |
| | | SKNTAYLQMNSLRAEDTAVYYCARGGFALDHWGQGT | |
| | | LVTVSS | |
| CD47-31 | CDR-L1 | DVHSKVA | 102 |
| | CDR-L2 | YSTNRLYS | 116 |
| | CDR-L3 | QQHSNYPVT | 63 |
| | VL | DIQMTQSPSSLSASVGDRVTITCRASQDVHSKVAWYQQ | 117 |
| | | KPGKAPKLLIYSTNRLYSGVPSRFSGSGSGTDFTLTISSL | |
| | | QPEDFATYYCQQHSNYPVTFGQGTKVEIKR | |
| | CDR-H1 | FTISNWWIH | 13 |
| | CDR-H2 | AINPFNGRTD | 118 |
| | CDR-H3 | TRGGYAMDY | 72 |
| | VH | EVQLVESGGGLVQPGGSLRLSCAASGFTISNWWIHWV | 119 |
| | | RQAPGKGLEWVAAINPFNGRTDYADSVKGRFTISADTS | |
| | | KNTAYLQMNSLRAEDTAVYYCTRGGYAMDYWGQGT | |
| | | LVTVSS | |
| CD47-32 | CDR-L1 | DVHYTVA | 120 |
| | CDR-L2 | YGTNRLYT | 54 |
| | CDR-L3 | QQHFNFPIT | 121 |
| | VL | DIQMTQSPSSLSASVGDRVTITCRASQDVHYTVAWYQ | 122 |
| | | QKPGKAPKLLIYGTNRLYTGVPSRFSGSGSGTDFTLTIS | |
| | | SLQPEDFATYYCQQHFNFPITFGQGTKVEIKR | |
| | CDR-H1 | FTINNFWIH | 123 |
| | CDR-H2 | NISPFTGDTR | 124 |
| | CDR-H3 | ARGGYAMDI | 59 |
| | VH | EVQLVESGGGLVQPGGSLRLSCAASGFTINNFWIHWVR | 125 |
| | | QAPGKGLEWVANISPFTGDTRYADSVKGRFTISADTSK | |
| | | NTAYLQMNSLRAEDTAVYYCARGGYAMDIWGQGTLV | |
| | | TVSS | |

According to the sequencing results, each of the selected scFvs had a light chain constant (CL) sequence of SEQ ID NO: 126, and a heavy chain constant (CH) sequence of SEQ ID NO: 127.

Preparation of Anti-CD47 IgGs

The anti-CD47 scFvs were respectively transferred to their IgG forms so as to increase the stability and for further applications. For light chain IgG construction, the signal peptide, variable domain and constant domains of IgG light chain were PCR assembled, and then ligated into a first mammalian cell expression vector to form a light chain plasmid. For heavy chain IgG construction, the signal peptide, variable domain and constant domain of IgG heavy chain were ligated into a second mammalian cell DNA vector to form a heavy chain plasmid. After co-transfecting CHO cells, the IgG forms of antibodies were collected from the culture medium and then purified by columns.

18 anti-CD47 IgGs were respectively produced from the anti-CD47 scFvs, and were respectively designated as “CD47-2 IgG”, “CD47-3 IgG”, “CD47-4 IgG”, “CD47-5 IgG”, “CD47-6 IgG”, “CD47-7 IgG”, “CD47-8 IgG”, “CD47-9 IgG”, “CD47-14 IgG”, “CD47-20 IgG”, “CD47-22 IgG”, “CD47-26 IgG”, “CD47-27 IgG”, “CD47-28 IgG”, “CD47-29 IgG”, “CD47-30 IgG”, “CD47-31 IgG” and “CD47-32 IgG”.

Antibody-Dependent Cellular Phagocytosis (ADCP) Analysis

To analyze the ADCP signal induction of the anti-CD47 IgGs, antibodies were 10-fold serial diluted on 96-well plate. Effector cells (human CD32a-167H expressing Jurkat cells integrated with the NFAT luciferase DNA fragment as reporter) and target cells (CHO-CD47 cells) were mixed. The ratio of effector cells and target cells was 3:1 (6×10⁴ cells/well:2×10⁴ cells/well). The cell mixtures were added into wells containing the specified anti-CD47 IgGs and incubated at 37° C. in a CO₂ incubator for 5 hours. Luminescence was measured by luciferase assay, and the results were expressed as relative luminescence units (RLU).

### Example 1 Characterization of Anti-CD47 scFvs

The data of FIG. 1 demonstrated that among the 30 selected scFvs, 18 scFvs exhibited a binding affinity to CD47 polypeptide. To analyze whether these anti-CD47 scFvs would be able to bind to the CD47 protein expressed on mammalian cell surface, DNA fragment comprising sequence encoding full-length CD47 was constructed into mammalian expression vector and transfected to CHO cells (a mammalian cell line without expressing human CD47 originally). A stable clone named as “CHO-CD47 cells”, which stably expressed CD47 on the surface thereof, was selected from the CD47-transfected cells (data not shown).

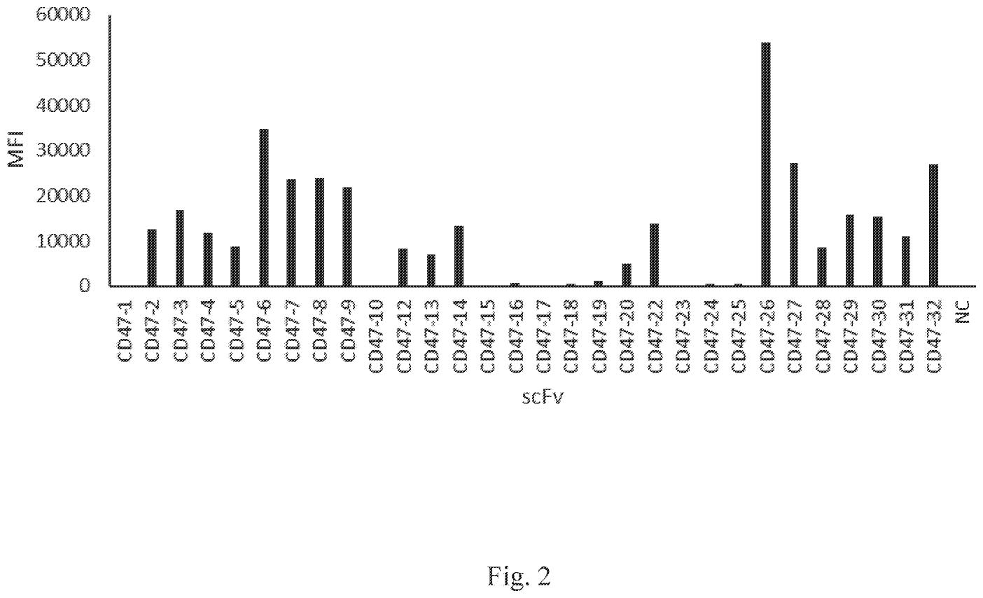

The binding of the anti-CD47 scFvs to the surface of CHO-CD47 cells was determined by flow cytometry, and the data was expressed as mean fluorescence intensity (MFI). NC represented as the treatment of unrelated scFv (i.e., scFv exhibiting no binding affinity to CD47; serving as a negative control in the study). The data of FIG. 2 indicated that compared to the negative control NC, 18 selected scFvs (including scFvs CD47-2, CD47-3, CD47-4, CD47-5, CD47-6, CD47-7, CD47-8, CD47-9, CD47-14, CD47-20, CD47-22, CD47-26, CD47-27, CD47-28, CD47-29, CD47-30, CD47-31 and CD47-32) recognized and bound to the CD47 polypeptide expressed on mammalian cells (CHO-CD47).

Various amounts of anti-CD47 scFvs were then mixed with CHO-CD47 cells at room temperature for one hour. Cells were washed and mixed with human SIRPα-mFc (100 ng) protein at 37° C. for 1 hour. Next, cells were washed and mixed with allophycocyanin (APC)-conjugated anti-mouse Fc antibody. The relative MFI was measured by flow cytometry and normalized to unrelated scFv (NC) as zero percent of blocking.

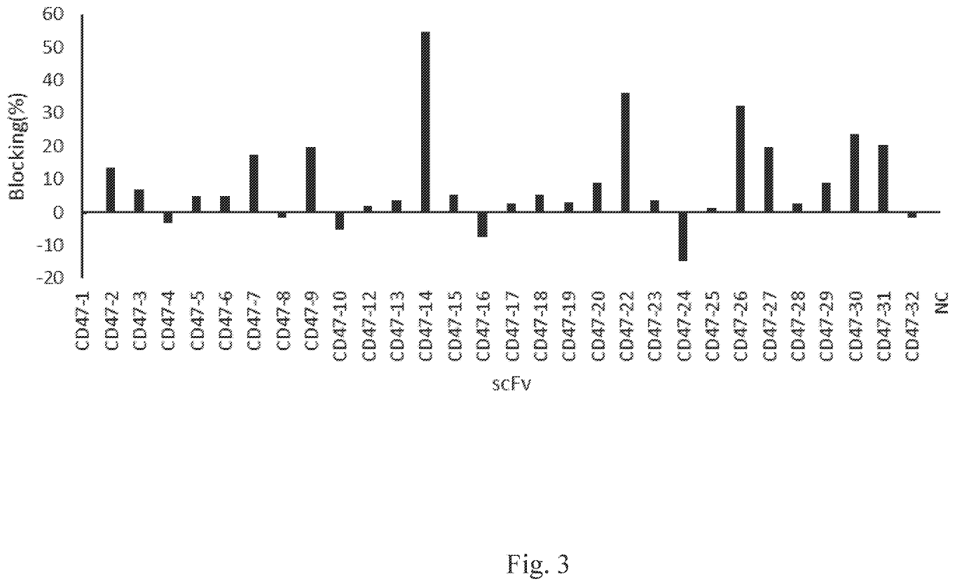

The data of FIG. 3 indicated that the present anti-CD47 scFvs blocked human SIRPα binding to CHO-CD47 cell at different levels, in which scFvs CD47-2, CD47-7, CD47-9, CD47-14, CD47-22, CD47-26, CD47-27, CD47-30 and CD47-31 exhibited superior activities to inhibit the binding of human SIRPα to CHO-CD47 cell surface as compared to other scFvs.

### Example 2 Characterization of Anti-CD47 IgGs

The anti-CD47 scFvs were respectively expressed in their IgG formats in accordance with the procedure described in Materials and Methods. CHO cells expressing CD47 (CHO-CD47 cells) and wild-type CHO cells (CHO-WT cells) were respectively treated with anti-CD47 IgGs. After washing, the binding of the anti-CD47 IgGs was detected using a polyclonal APC-conjugated anti-human IgG antibody. Relative MFI was measured by flow cytometry. Antibody R23, a reference antibody constructed according to the sequences of ADI29341 disclosed in US20200181259A1, served as a positive control in this study.

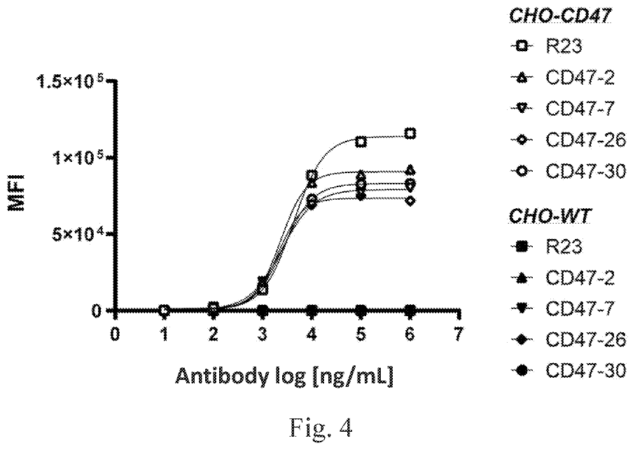

According to the analytic results, CD47-2, CD47-7, CD47-26 and CD47-30 IgGs efficiently bound to cellular CD47 at low concentration (FIG. 4). The data of Table 2 further confirmed that the EC50 of CD47-2, CD47-7, CD47-26 and CD47-30 IgGs was lower than that of antibody R23, indicated that CD47-2, CD47-7, CD47-26 and CD47-30 IgGs is useful in detecting CD47 expression and/or blocking CD47-mediated signal pathway.

TABLE 2
EC50 of specified anti-CD47 IgGs

| primary antibody | EC50 (ng/mL) |
|---|---|
| R23 | 4,178 |
| CD47-2 | 2,337 |
| CD47-7 | 2,442 |
| CD47-26 | 2,050 |
| CD47-30 | 2,673 |

Various amounts of anti-CD47 IgGs were then mixed with CHO-CD47 cells at room temperature for one hour. Cells were washed and mixed with human SIRPα-mFc (500 ng) protein at 37° C. for 1 hour. Next, cells were washed followed by mixing with APC conjugated anti-mouse Fc antibody. The relative MFI was measured by flow cytometry and normalized to unrelated IgG (NC1 and NC2) as zero percent of blocking. R23 served as a positive control in this study.

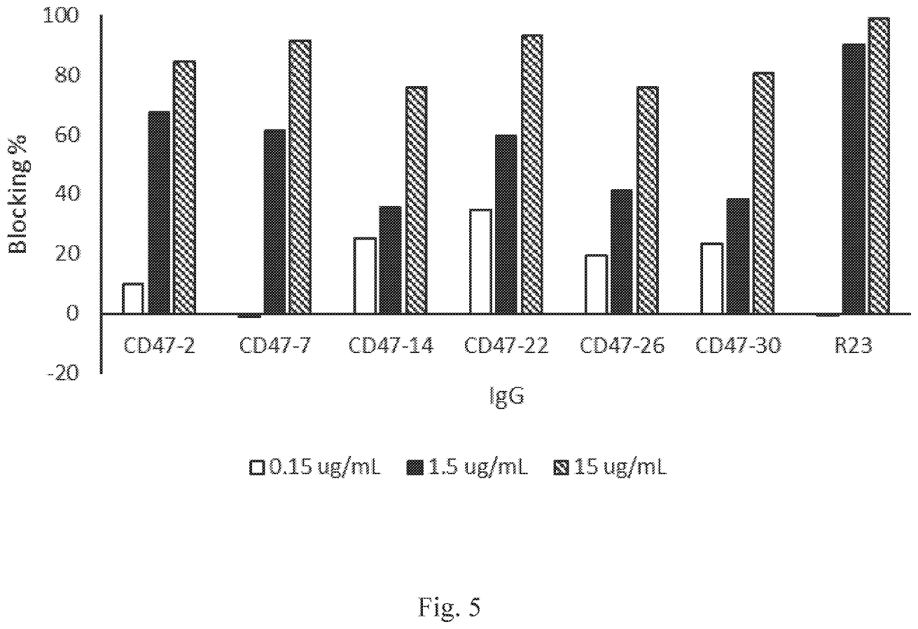

The data of FIG. 5 demonstrated that the present CD47-2, CD47-7, CD47-14, CD46-22, CD47-26 and CD47-30 IgGs (at a concentration of 0.15 μg/ml, 1.5 μg/ml or 15 μg/ml) could efficiently block human SIRPα binding to CHO-CD47 cells.

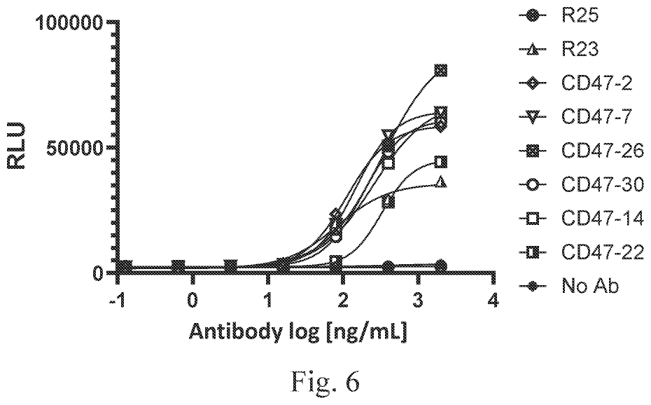

The data of FIG. 6 demonstrated that the present CD47-2, CD47-7, CD47-14, CD46-22, CD47-26 and CD47-30 IgGs can induce stronger ADCP signals than R23 reference antibody toward CD47 over-expression cell (CHO-CD47).

In conclusion, the present disclosure provides 18 recombinant antibodies, which exhibited binding affinity and block activity toward CD47 polypeptide. The present recombinant antibodies provide a potential means to treat different CD47-related diseases, especially the diseases caused by and/or associated with CD47 overexpression, via inhibiting the negative regulation of CD47 signaling thereby enhancing the immune response against cancer cells and/or pathogens.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant antibody or a fragment thereof, comprising a variable light chain (VL) domain and a variable heavy chain (VH) domain, wherein the VL domain comprises a first light chain complementarity determining region (CDR-L1), a second light chain CDR (CDR-L2) and a third light chain CDR (CDR-L3), and the VH domain comprises a first heavy chain CDR (CDR-H1), a second heavy chain CDR (CDR-H2) and a third heavy chain CDR (CDR-H3), wherein
the CDR-L1, CDR-L2, CDR-L3, CDR-H1, CDR-H2 and CDR-H3 respectively comprise the amino acid sequences of SEQ ID NOs: 1, 2, 3, 5, 6 and 7;
the CDR-L1, CDR-L2, CDR-L3, CDR-H1, CDR-H2 and CDR-H3 respectively comprise the amino acid sequences of SEQ ID NOs: 9, 10, 11, 13, 14 and 15;
the CDR-L1, CDR-L2, CDR-L3, CDR-H1, CDR-H2 and CDR-H3 respectively comprise the amino acid sequences of SEQ ID NOs: 17, 18, 19, 21, 22 and 23;
the CDR-L1, CDR-L2, CDR-L3, CDR-H1, CDR-H2 and CDR-H3 respectively comprise the amino acid sequences of SEQ ID NOs: 25, 18, 26, 28, 29 and 30;
the CDR-L1, CDR-L2, CDR-L3, CDR-H1, CDR-H2 and CDR-H3 respectively comprise the amino acid sequences of SEQ ID NOs: 32, 33, 26, 35, 36 and 37;
the CDR-L1, CDR-L2, CDR-L3, CDR-H1, CDR-H2 and CDR-H3 respectively comprise the amino acid sequences of SEQ ID NOs: 39, 40, 41, 43, 44 and 45;
the CDR-L1, CDR-L2, CDR-L3, CDR-H1, CDR-H2 and CDR-H3 respectively comprise the amino acid sequences of SEQ ID NOs: 47, 2, 48, 50, 51 and 52;
the CDR-L1, CDR-L2, CDR-L3, CDR-H1, CDR-H2 and CDR-H3 respectively comprise the amino acid sequences of SEQ ID NOs: 9, 54, 55, 57, 58 and 59;
the CDR-L1, CDR-L2, CDR-L3, CDR-H1, CDR-H2 and CDR-H3 respectively comprise the amino acid sequences of SEQ ID NOs: 61, 62, 63, 13, 65 and 66;
the CDR-L1, CDR-L2, CDR-L3, CDR-H1, CDR-H2 and CDR-H3 respectively comprise the amino acid sequences of SEQ ID NOs: 25, 33, 68, 70, 71 and 72;
the CDR-L1, CDR-L2, CDR-L3, CDR-H1, CDR-H2 and CDR-H3 respectively comprise the amino acid sequences of SEQ ID NOs: 74, 75, 76, 78, 79 and 80;
the CDR-L1, CDR-L2, CDR-L3, CDR-H1, CDR-H2 and CDR-H3 respectively comprise the amino acid sequences of SEQ ID NOs: 82, 83, 84, 86, 87 and 72;
the CDR-L1, CDR-L2, CDR-L3, CDR-H1, CDR-H2 and CDR-H3 respectively comprise the amino acid sequences of SEQ ID NOs: 89, 90, 91, 93, 94 and 66;
the CDR-L1, CDR-L2, CDR-L3, CDR-H1, CDR-H2 and CDR-H3 respectively comprise the amino acid sequences of SEQ ID NOs: 96, 97, 98, 35, 100 and 72;
the CDR-L1, CDR-L2, CDR-L3, CDR-H1, CDR-H2 and CDR-H3 respectively comprise the amino acid sequences of SEQ ID NOs: 102, 103, 68, 35, 105 and 106;
the CDR-L1, CDR-L2, CDR-L3, CDR-H1, CDR-H2 and CDR-H3 respectively comprise the amino acid sequences of SEQ ID NOs: 108, 109, 110, 112, 113 and 114;
the CDR-L1, CDR-L2, CDR-L3, CDR-H1, CDR-H2 and CDR-H3 respectively comprise the amino acid sequences of SEQ ID NOs: 102, 116, 63, 13, 118 and 72; or
the CDR-L1, CDR-L2, CDR-L3, CDR-H1, CDR-H2 and CDR-H3 respectively comprise the amino acid sequences of SEQ ID NOs: 120, 54, 121, 123, 124 and 59.

2. The recombinant antibody of claim 1, wherein
the CDR-L1, CDR-L2, CDR-L3, CDR-H1, CDR-H2 and CDR-H3 respectively comprise the amino acid sequences of SEQ ID NOs: 1, 2, 3, 5, 6 and 7;
the CDR-L1, CDR-L2, CDR-L3, CDR-H1, CDR-H2 and CDR-H3 respectively comprise the amino acid sequences of SEQ ID NOs: 39, 40, 41, 43, 44 and 45;
the CDR-L1, CDR-L2, CDR-L3, CDR-H1, CDR-H2 and CDR-H3 respectively comprise the amino acid sequences of SEQ ID NOs: 61, 62, 63, 13, 65 and 66;
the CDR-L1, CDR-L2, CDR-L3, CDR-H1, CDR-H2 and CDR-H3 respectively comprise the amino acid sequences of SEQ ID NOs: 74, 75, 76, 78, 79 and 80;
the CDR-L1, CDR-L2, CDR-L3, CDR-H1, CDR-H2 and CDR-H3 respectively comprise the amino acid sequences of SEQ ID NOs: 82, 83, 84, 86, 87 and 72; or
the CDR-L1, CDR-L2, CDR-L3, CDR-H1, CDR-H2 and CDR-H3 respectively comprise the amino acid sequences of SEQ ID NOs: 108, 109, 110, 112, 113 and 114.

3. The recombinant antibody of claim 1, wherein
the VL and VH domains respectively comprise the amino acid sequences of SEQ ID NOs: 4 and 8;
the VL and VH domains respectively comprise the amino acid sequences of SEQ ID NOs: 12 and 16;
the VL and VH domains respectively comprise the amino acid sequences of SEQ ID NOs: 20 and 24;
the VL and VH domains respectively comprise the amino acid sequences of SEQ ID NOs: 27 and 31;
the VL and VH domains respectively comprise the amino acid sequences of SEQ ID NOs: 34 and 38;
the VL and VH domains respectively comprise the amino acid sequences of SEQ ID NOs: 42 and 46;
the VL and VH domains respectively comprise the amino acid sequences of SEQ ID NOs: 49 and 53;
the VL and VH domains respectively comprise the amino acid sequences of SEQ ID NOs: 56 and 60;
the VL and VH domains respectively comprise the amino acid sequences of SEQ ID NOs: 64 and 67;
the VL and VH domains respectively comprise the amino acid sequences of SEQ ID NOs: 69 and 73;
the VL and VH domains respectively comprise the amino acid sequences of SEQ ID NOs: 77 and 81;
the VL and VH domains respectively comprise the amino acid sequences of SEQ ID NOs: 85 and 88;
the VL and VH domains respectively comprise the amino acid sequences of SEQ ID NOs: 92 and 95;
the VL and VH domains respectively comprise the amino acid sequences of SEQ ID NOs: 99 and 101;
the VL and VH domains respectively comprise the amino acid sequences of SEQ ID NOs: 104 and 107;
the VL and VH domains respectively comprise the amino acid sequences of SEQ ID NOs: 111 and 115;
the VL and VH domains respectively comprise the amino acid sequences of SEQ ID NOs: 117 and 119; or
the VL and VH domains respectively comprise the amino acid sequences of SEQ ID NOs: 122 and 125.

4. The recombinant antibody of claim 3, wherein
the VL and VH domains respectively comprise the amino acid sequences of SEQ ID NOs: 4 and 8;
the VL and VH domains respectively comprise the amino acid sequences of SEQ ID NOs: 42 and 46;
the VL and VH domains respectively comprise the amino acid sequences of SEQ ID NOs: 64 and 67;
the VL and VH domains respectively comprise the amino acid sequences of SEQ ID NOs: 77 and 81;
the VL and VH domains respectively comprise the amino acid sequences of SEQ ID NOs: 85 and 88; or
the VL and VH domains respectively comprise the amino acid sequences of SEQ ID NOs: 111 and 115.

5. A pharmaceutical composition, comprising the recombinant antibody of claim 1 and a pharmaceutically acceptable carrier.

6. A method of treating a CD47-related disease in a subject, comprising administering to the subject an effective amount of the pharmaceutical composition of claim 5.

7. The method of claim 6, wherein the CD47-related disease is a cancer or an infectious disease.

8. The method of claim 7, wherein the cancer is a CD47-expressing cancer.

9. The method of claim 8, wherein the CD47-expressing cancer is a head and neck cancer, breast cancer, small cell lung cancer (SCLC), non-small cell lung cancer (NSCLC), liver cancer, gastric cancer, pancreatic cancer, colorectal cancer, ovarian cancer, bladder cancer, cholangiocarcinoma, glioblastoma, osteosarcoma, leiomyosarcoma (LMS), non-Hodgkin's lymphoma, acute myeloid leukemia (AML), acute lymphoblastic leukemia (ALL), chronic myeloid leukemia (CML), myelodysplastic syndrome (MDS), or multiple myeloma (MM).

10. The method of claim 6, wherein the subject is a human.


SEQUENCE LISTING (136 sequences)

SEQ ID NO: 1 - protein, 7 aa, Artificial Sequence
  synthesized_CD47-2_CDR-L1
DVSSKVA

SEQ ID NO: 2 - protein, 8 aa, Artificial Sequence
  synthesized_CD47-2_CDR-L2
YGTNRLYP

SEQ ID NO: 3 - protein, 9 aa, Artificial Sequence
  synthesized_CD47-2_CDR-L3
QQSSNYPLT

SEQ ID NO: 4 - protein, 108 aa, Artificial Sequence
  synthesized_CD47-2_VL
DIQMTQSPSSLSASVGDRVTITCRASQDVSSKVAWYQQKPGKAPKLLIYGTNRLYPGVPSRFSGSGSGTDFTLTISSLQPEDFATYYCQQSSNYPLTFGQGTKVEIKR

SEQ ID NO: 5 - protein, 9 aa, Artificial Sequence
  synthesized_CD47-2_CDR-H1
FTISNSFIH

SEQ ID NO: 6 - protein, 10 aa, Artificial Sequence
  synthesized_CD47-2_CDR-H2
YIWPWSGSTG

SEQ ID NO: 7 - protein, 9 aa, Artificial Sequence
  synthesized_CD47-2_CDR-H3
ARGGFSMDH

SEQ ID NO: 8 - protein, 116 aa, Artificial Sequence
  synthesized_CD47-2_VH
EVQLVESGGGLVQPGGSLRLSCAASGFTISNSFIHWVRQAPGKGLEWVAYIWPWSGSTGYADSVKGRFTISADTSKNTAYLQMNSLRAEDTAVYYCARGGFSMDHWGQGTLVTVSS

SEQ ID NO: 9 - protein, 7 aa, Artificial Sequence
  synthesized_CD47-3_CDR-L1
DVSNRVA

SEQ ID NO: 10 - protein, 8 aa, Artificial Sequence
  synthesized_CD47-3_CDR-L2
SRANGLAS

SEQ ID NO: 11 - protein, 9 aa, Artificial Sequence
  synthesized_CD47-3_CDR-L3
QQGSHFPIT

SEQ ID NO: 12 - protein, 108 aa, Artificial Sequence
  synthesized_CD47-3_VL
DIQMTQSPSSLSASVGDRVTITCRASQDVSNRVAWYQQKPGKAPKLLISRANGLASGVPSRFSGSGSGTDFTLTISSLQPEDFATYYCQQGSHFPITFGQGTKVEIKR

SEQ ID NO: 13 - protein, 9 aa, Artificial Sequence
  synthesized_CD47-3_CDR-H1
FTISNWWIH

SEQ ID NO: 14 - protein, 10 aa, Artificial Sequence
  synthesized_CD47-3_CDR-H2
HIGPTVGKTY

SEQ ID NO: 15 - protein, 9 aa, Artificial Sequence
  synthesized_CD47-3_CDR-H3
ARGGFAMDH

SEQ ID NO: 16 - protein, 116 aa, Artificial Sequence
  synthesized_CD47-3_VH
EVQLVESGGGLVQPGGSLRLSCAASGFTISNWWIHWVRQAPGKGLEWVAHIGPTVGKTYYADSVKGRFTISADTSKNTAYLQMNSLRAEDTAVYYCARGGFAMDHWGQGTLVTVSS

SEQ ID NO: 17 - protein, 7 aa, Artificial Sequence
  synthesized_CD47-4_CDR-L1
SVDNGVA

SEQ ID NO: 18 - protein, 8 aa, Artificial Sequence
  synthesized_CD47-4_CDR-L2
YSATRLYP

SEQ ID NO: 19 - protein, 9 aa, Artificial Sequence
  synthesized_CD47-4_CDR-L3
QQSSTFPLT

SEQ ID NO: 20 - protein, 108 aa, Artificial Sequence
  synthesized_CD47-4_VL
DIQMTQSPSSLSASVGDRVTITCRASQSVDNGVAWYQQKPGKAPKLLIYSATRLYPGVLSRFSGSGSGTDFTLTISSLQPEDFATYYCQQSSTFPLTFGQGTKVEIKR

SEQ ID NO: 21 - protein, 9 aa, Artificial Sequence
  synthesized_CD47-4_CDR-H1
FTINDYWIH

SEQ ID NO: 22 - protein, 10 aa, Artificial Sequence
  synthesized_CD47-4_CDR-H2
HIWPTYGGTH

SEQ ID NO: 23 - protein, 9 aa, Artificial Sequence
  synthesized_CD47-4_CDR-H3
ARGGYSLDI

SEQ ID NO: 24 - protein, 116 aa, Artificial Sequence
  synthesized_CD47-4_VH
EVQLVESGGGLVQPGGSLRLSCAASGFTINDYWIHWVRQAPGKGLEWVAHIWPTYGGTHYADSVKGRFTISADTSKNTAYLQMNSLRAEDAAVYYCARGGYSLDIWGQGTLVTVSS

SEQ ID NO: 25 - protein, 7 aa, Artificial Sequence
  synthesized_CD47-5_CDR-L1
DVHNKVA

SEQ ID NO: 26 - protein, 9 aa, Artificial Sequence
  synthesized_CD47-5_CDR-L3
QQHFNYPLT

SEQ ID NO: 27 - protein, 108 aa, Artificial Sequence
  synthesized_CD47-5_VL
DIQMTQSPSSLSASVGDRVTITCRASQDVHNKVAWYQQKPGKAPKLLIYSATRLYPGVPSRFSGSGSGTDFTLTISSLQPEDFATYYCQQHFNYPLTFGQGTKVEIKR

SEQ ID NO: 28 - protein, 9 aa, Artificial Sequence
  synthesized_CD47-5_CDR-H1
FTITNYWIH

SEQ ID NO: 29 - protein, 10 aa, Artificial Sequence
  synthesized_CD47-5_CDR-H2
HIWPGVGDTD

SEQ ID NO: 30 - protein, 9 aa, Artificial Sequence
  synthesized_CD47-5_CDR-H3
ARGGFAMDL

SEQ ID NO: 31 - protein, 116 aa, Artificial Sequence
  synthesized_CD47-5_VH
EVQLVESGGGLVQPGGSLRLSCAASGFTITNYWIHWVRQAPGKGLEWVAHIWPGVGDTDYADSVKGRFTISADTSKNTAYLQMNSLRAEDTAVYYCARGGFAMDLWGQGTLVTVSS

SEQ ID NO: 32 - protein, 7 aa, Artificial Sequence
  synthesized_CD47-6_CDR-L1
DVSSRVA

SEQ ID NO: 33 - protein, 8 aa, Artificial Sequence
  synthesized_CD47-6_CDR-L2
YYTSNLYS

SEQ ID NO: 34 - protein, 108 aa, Artificial Sequence
  synthesized_CD47-6_VL
DIQMTQSPSSLSASVGDRVTITCRASQDVSSRVAWYQQKPGKAPKLLIYYTSNLYSGVPSRFSGSGSGTDFTLTISSLQPEDFATYYCQQHFNYPLTFGQGTKVEIKR

SEQ ID NO: 35 - protein, 9 aa, Artificial Sequence
  synthesized_CD47-6_CDR-H1
FTITNWWIH

SEQ ID NO: 36 - protein, 10 aa, Artificial Sequence
  synthesized_CD47-6_CDR-H2
TISPYSGDTH

SEQ ID NO: 37 - protein, 9 aa, Artificial Sequence
  synthesized_CD47-6_CDR-H3
ARGGYAMDH

SEQ ID NO: 38 - protein, 116 aa, Artificial Sequence
  synthesized_CD47-6_VH
EVQLVESGGGLVQPGGSLRLSCAASGFTITNWWIHWVRQAPGKGLEWVATISPYSGDTHYADSVKGRFTISADTSKNTAYLQMNSLRAEDTAVYYCARGGYAMDHWGQGTLVTVSS

SEQ ID NO: 39 - protein, 7 aa, Artificial Sequence
  synthesized_CD47-7_CDR-L1
DVYTRVA

SEQ ID NO: 40 - protein, 8 aa, Artificial Sequence
  synthesized_CD47-7_CDR-L2
YYTRNLYS

SEQ ID NO: 41 - protein, 9 aa, Artificial Sequence
  synthesized_CD47-7_CDR-L3
QQAANYPLT

SEQ ID NO: 42 - protein, 108 aa, Artificial Sequence
  synthesized_CD47-7_VL
DIQMTQSPSSLSASVGDRVTITCRASQDVYTRVAWYQQKPGKAPKLLIYYTRNLYSGVPSRFSGSGSGTDFTLTISSLQPEDFATYYCQQAANYPLTFGQGTKVEIKR

SEQ ID NO: 43 - protein, 8 aa, Artificial Sequence
  synthesized_CD47-7_CDR-H1
TITNYYIH

SEQ ID NO: 44 - protein, 10 aa, Artificial Sequence
  synthesized_CD47-7_CDR-H2
SIWPGYGNTG

SEQ ID NO: 45 - protein, 9 aa, Artificial Sequence
  synthesized_CD47-7_CDR-H3
ARGGYSMDF

SEQ ID NO: 46 - protein, 116 aa, Artificial Sequence
  synthesized_CD47-7_VH
EVQLVESGGGLVQPGGSLRLSCAASGFTITNYYIHWVRQAPGKGLEWVASIWPGYGNTGYADSVKGRFTISADTSKNTAYLQMNSLRAEDTAVYYCARGGYSMDFWGQGTLVTVSS

SEQ ID NO: 47 - protein, 7 aa, Artificial Sequence
  synthesized_CD47-8_CDR-L1
DVHYAVA

SEQ ID NO: 48 - protein, 9 aa, Artificial Sequence
  synthesized_CD47-8_CDR-L3
QQHSNFPLT

SEQ ID NO: 49 - protein, 108 aa, Artificial Sequence
  synthesized_CD47-8_VL
DIQMTQSPSSLSASVGDRVTITCRASQDVHYAVAWYQQKPGKAPKLLIYGTNRLYPGVPSRFSGSGSGTDFTLTISSLQPEDFATYYCQQHSNFPLTFGQGTKVEIKR

SEQ ID NO: 50 - protein, 9 aa, Artificial Sequence
  synthesized_CD47-8_CDR-H1
FTINNFYIH

SEQ ID NO: 51 - protein, 10 aa, Artificial Sequence
  synthesized_CD47-8_CDR-H2
SIWPWSGETG

SEQ ID NO: 52 - protein, 9 aa, Artificial Sequence
  synthesized_CD47-8_CDR-H3
ARGGFAMDF

SEQ ID NO: 53 - protein, 116 aa, Artificial Sequence
  synthesized_CD47-8_VH
EVQLVESGGGLVQPGGSLRLSCAASGFTINNFYIHWVRQAPGKGLEWVASIWPWSGETGYADSVKGRFTIGADTSKNTAYLQMNSLRAEDTAVYYCARGGFAMDFWGQGTLVTVSS

SEQ ID NO: 54 - protein, 8 aa, Artificial Sequence
  synthesized_CD47-9_CDR-L2
YGTNRLYT

SEQ ID NO: 55 - protein, 9 aa, Artificial Sequence
  synthesized_CD47-9_CDR-L3
QQHFNYPIT

SEQ ID NO: 56 - protein, 108 aa, Artificial Sequence
  synthesized_CD47-9_VL
DIQMTQSPSSLSASVGDRVTITCRASQDVSNRVAWYQQKPGKAPKLLIYGTNRLYTGVPSRFSGSGSGTDFTLTISSLQPEDFATYYCQQHFNYPITFGQGTKVEIKR

SEQ ID NO: 57 - protein, 9 aa, Artificial Sequence
  synthesized_CD47-9_CDR-H1
FTINSWWIH

SEQ ID NO: 58 - protein, 10 aa, Artificial Sequence
  synthesized_CD47-9_CDR-H2
KISPFNGDTH

SEQ ID NO: 59 - protein, 9 aa, Artificial Sequence
  synthesized_CD47-9_CDR-H3
ARGGYAMDI

SEQ ID NO: 60 - protein, 116 aa, Artificial Sequence
  synthesized_CD47-9_VH
EVQLVESGGGLVQPGGSLRLSCAASGFTINSWWIHWVRQAPGKGLEWVAKISPFNGDTHYADSVKGRFTISADTSKNTAYLQMNSLRAEDTAVYYCARGGYAMDIWGQGTLVTVSS

SEQ ID NO: 61 - protein, 7 aa, Artificial Sequence
  synthesized_CD47-14_CDR-L1
DVSTKVA

SEQ ID NO: 62 - protein, 8 aa, Artificial Sequence
  synthesized_CD47-14_CDR-L2
YFTRNLYS

SEQ ID NO: 63 - protein, 9 aa, Artificial Sequence
  synthesized_CD47-14_CDR-L3
QQHSNYPVT

SEQ ID NO: 64 - protein, 108 aa, Artificial Sequence
  synthesized_CD47-14_VL
DIQMTQSPSSLSASVGDRVTITCRASQDVSTKVAWYQQKPGKAPKLLIYFTRNLYSGVPSRFSGSGSGTDFTLTISSLQPEDFATYYCQQHSNYPVTFGQGTKVEIKR

SEQ ID NO: 65 - protein, 10 aa, Artificial Sequence
  synthesized_CD47-14_CDR-H2
GISPYSGKTD

SEQ ID NO: 66 - protein, 9 aa, Artificial Sequence
  synthesized_CD47-14_CDR-H3
ARGGYAMDL

SEQ ID NO: 67 - protein, 116 aa, Artificial Sequence
  synthesized_CD47-14_VH
EVQLVESGGGLVQPGGSLRLSCAASGFTISNWWIHWVRQAPGKGLEWVAGISPYSGKTDYADSVKGRFTISADTSKNTAYLQMNSLRAEDTAVYYCARGGYAMDLWGQGTLVTVSS

SEQ ID NO: 68 - protein, 9 aa, Artificial Sequence
  synthesized_CD47-20_CDR-L3
QQHFTYPLT

SEQ ID NO: 69 - protein, 108 aa, Artificial Sequence
  synthesized_CD47-20_VL
DIQMTQSPSSLSASVGDRVTITCRASQDVHNKVAWYQQKPGKAPKLLIYYTSNLYSGVPSRFSGSGSGTDFTLTISSLQPEDFATYYCQQHFTYPLTFGQGTKVEIKR

SEQ ID NO: 70 - protein, 9 aa, Artificial Sequence
  synthesized_CD47-20_CDR-H1
FTISNYYIH

SEQ ID NO: 71 - protein, 10 aa, Artificial Sequence
  synthesized_CD47-20_CDR-H2
GIWPSGGSTS

SEQ ID NO: 72 - protein, 9 aa, Artificial Sequence
  synthesized_CD47-20_CDR-H3
TRGGYAMDY

SEQ ID NO: 73 - protein, 116 aa, Artificial Sequence
  synthesized_CD47-20_VH
EVQLVESGGGLVQPGGSLRLSCAASGFTISNYYIHWVRQAPGKGLEWVAGIWPSGGSTSYADSVKGRFTISADTSKNTAYLQMNSLRAEDTAVYYCTRGGYAMDYWGQGTLVTVSS

SEQ ID NO: 74 - protein, 7 aa, Artificial Sequence
  synthesized_CD47-22_CDR-L1
DVHSWVA

SEQ ID NO: 75 - protein, 8 aa, Artificial Sequence
  synthesized_CD47-22_CDR-L2
SRATWLAS

SEQ ID NO: 76 - protein, 9 aa, Artificial Sequence
  synthesized_CD47-22_CDR-L3
QQSSNFPLT

SEQ ID NO: 77 - protein, 108 aa, Artificial Sequence
  synthesized_CD47-22_VL
DIQMTQSPSSLSASVGDRVTITCRASQDVHSWVAWYQQKPGKAPKLLISRATWLASGVPSRFSGSGSGTDFTLTISSLQPEDFATYYCQQSSNFPLTFGQGTKVEIKR

SEQ ID NO: 78 - protein, 9 aa, Artificial Sequence
  synthesized_CD47-22_CDR-H1
FTINDYGIH

SEQ ID NO: 79 - protein, 10 aa, Artificial Sequence
  synthesized_CD47-22_CDR-H2
GIGPFWGSTF

SEQ ID NO: 80 - protein, 13 aa, Artificial Sequence
  synthesized_CD47-22_CDR-H3
ARWMWGRWSAMDY

SEQ ID NO: 81 - protein, 120 aa, Artificial Sequence
  synthesized_CD47-22_VH
EVQLVESGGGLVQPGGSLRLSCAASGFTINDYGIHWVRQAPGKGLEWVAGIGPFWGSTFYADSVKGRFTISADTSKNTAYLQMNSLRAEDTAVYYCARWMWGRWSAMDYWGQGTLVTVSS

SEQ ID NO: 82 - protein, 7 aa, Artificial Sequence
  synthesized_CD47-26_CDR-L1
DVSNYVA

SEQ ID NO: 83 - protein, 8 aa, Artificial Sequence
  synthesized_CD47-26_CDR-L2
YGTSRLYP

SEQ ID NO: 84 - protein, 9 aa, Artificial Sequence
  synthesized_CD47-26_CDR-L3
QQHSSYPLT

SEQ ID NO: 85 - protein, 108 aa, Artificial Sequence
  synthesized_CD47-26_VL
DIQMTQSPSSLSASVGDRVTITCRASQDVSNYVAWYQQKPGKAPKLLIYGTSRLYPGVPSRFSGSGSGTDFTLTISSLQPEDFATYYCQQHSSYPLTFGQGTKVEIKR

SEQ ID NO: 86 - protein, 9 aa, Artificial Sequence
  synthesized_CD47-26_CDR-H1
FTITNFYIH

SEQ ID NO: 87 - protein, 10 aa, Artificial Sequence
  synthesized_CD47-26_CDR-H2
KITPYNGDTH

SEQ ID NO: 88 - protein, 116 aa, Artificial Sequence
  synthesized_CD47-26_VH
EVQLVESGGGLVQPGGSLRLSCAASGFTITNFYIHWVRQAPGKGLEWVAKITPYNGDTHYADSVKGRFTISADTSKNTAYLQMNSLRAEDTAVYYCTRGGYAMDYWGQGTLVTVSS

SEQ ID NO: 89 - protein, 7 aa, Artificial Sequence
  synthesized_CD47-27_CDR-L1
DVHNGVA

SEQ ID NO: 90 - protein, 8 aa, Artificial Sequence
  synthesized_CD47-27_CDR-L2
FRTSWLLS

SEQ ID NO: 91 - protein, 9 aa, Artificial Sequence
  synthesized_CD47-27_CDR-L3
QQGSSFPFT

SEQ ID NO: 92 - protein, 108 aa, Artificial Sequence
  synthesized_CD47-27_VL
DIQMTQSPSSLSASVGDRVTITCRASQDVHNGVAWYQQKPGKAPKLLIFRTSWLLSGVPSRFSGSGSGTDFTLTISSLQPEDFATYYCQQGSSFPFTFGQGTKVEIKR

SEQ ID NO: 93 - protein, 9 aa, Artificial Sequence
  synthesized_CD47-27_CDR-H1
FTINNYWIH

SEQ ID NO: 94 - protein, 10 aa, Artificial Sequence
  synthesized_CD47-27_CDR-H2
RISPFDGGTY

SEQ ID NO: 95 - protein, 116 aa, Artificial Sequence
  synthesized_CD47-27_VH
EVQLVESGGGLVQPGGSLRLSCAASGFTINNYWIHWVRQAPGKGLEWVARISPFDGGTYYADSVKGRFTISADTSKNTAYLQMNSLRAEDTAVYYCARGGYAMDLWGQGTLVTVSS

SEQ ID NO: 96 - protein, 7 aa, Artificial Sequence
  synthesized_CD47-28_CDR-L1
DVHGKVA

SEQ ID NO: 97 - protein, 8 aa, Artificial Sequence
  synthesized_CD47-28_CDR-L2
YYARNLYS

SEQ ID NO: 98 - protein, 9 aa, Artificial Sequence
  synthesized_CD47-28_CDR-L3
QQYFNFPLT

SEQ ID NO: 99 - protein, 108 aa, Artificial Sequence
  synthesized_CD47-28_VL
DIQMTQSPSSLSASVGDRVTITCRASQDVHGKVAWYQQKPGKAPKLLIYYARNLYSGVPSRFSGSGSGTDFTLTISSLQPEDFASYYCQQYFNFPLTFGQGTKVEIKR

SEQ ID NO: 100 - protein, 10 aa, Artificial Sequence
  synthesized_CD47-28_CDR-H2
EINPFGGNTY

SEQ ID NO: 101 - protein, 116 aa, Artificial Sequence
  synthesized_CD47-28_VH
EVQLVESGGGLVQPGGSLRLSCAASGFTITNWWIHWVRQAPGKGLEWVAEINPFGGNTYYADSVKGRFTISADTSKNTAYLQMNSLRAEDTAVYYCTRGGYAMDYWGQGTLVTVSS

SEQ ID NO: 102 - protein, 7 aa, Artificial Sequence
  synthesized_CD47-29_CDR-L1
DVHSKVA

SEQ ID NO: 103 - protein, 8 aa, Artificial Sequence
  synthesized_CD47-29_CDR-L2
YSTNRLYP

SEQ ID NO: 104 - protein, 108 aa, Artificial Sequence
  synthesized_CD47-29_VL
DIQMTQSPSSLSASVGDRVTITCRASQDVHSKVAWYQQKPGKAPKLLIYSTNRLYPGVPSRFSGSGSGTDFTLTISSLQPEDFATYYCQQHFTYPLTFGQGTKVEIKR

SEQ ID NO: 105 - protein, 10 aa, Artificial Sequence
  synthesized_CD47-29_CDR-H2
GITPYSGDTY

SEQ ID NO: 106 - protein, 9 aa, Artificial Sequence
  synthesized_CD47-29_CDR-H3
ARGGYAIDN

SEQ ID NO: 107 - protein, 116 aa, Artificial Sequence
  synthesized_CD47-29_VH
EVQLVESGGGLVQPGGSLRLSCAASGFTITNWWIHWVRQAPGKGLEWVAGITPYSGDTYYADSVKGRFTISADTSKNTAYLQMNSLRAEDTAVYYCARGGYAIDNWGQGTLVTVSS

SEQ ID NO: 108 - protein, 7 aa, Artificial Sequence
  synthesized_CD47-30_CDR-L1
DVSHYVA

SEQ ID NO: 109 - protein, 8 aa, Artificial Sequence
  synthesized_CD47-30_CDR-L2
YSTSRLYP

SEQ ID NO: 110 - protein, 9 aa, Artificial Sequence
  synthesized_CD47-30_CDR-L3
QQHSSYPIT

SEQ ID NO: 111 - protein, 108 aa, Artificial Sequence
  synthesized_CD47-30_VL
DIQMTQSPSSLSASVGDRVTITCRASQDVSHYVAWYQQKPGKAPKLLIYSTSRLYPGVPSRFSGSGSGTDFTLTISSLQPEDFATYYCQQHSSYPITFGQGTKVEIKR

SEQ ID NO: 112 - protein, 9 aa, Artificial Sequence
  synthesized_CD47-30_CDR-H1
FTINNWWIH

SEQ ID NO: 113 - protein, 10 aa, Artificial Sequence
  synthesized_CD47-30_CDR-H2
WIDPFNGDSD

SEQ ID NO: 114 - protein, 9 aa, Artificial Sequence
  synthesized_CD47-30_CDR-H3
ARGGFALDH

SEQ ID NO: 115 - protein, 116 aa, Artificial Sequence
  synthesized_CD47-30_VH
EVQLVESGGGLVQPGGSLRLSCAASGFTINNWWIHWVRQAPGKGLEWVAWIDPFNGDSDYADSVKGRFTISADTSKNTAYLQMNSLRAEDTAVYYCARGGFALDHWGQGTLVTVSS

SEQ ID NO: 116 - protein, 8 aa, Artificial Sequence
  synthesized_CD47-31_CDR-L2
YSTNRLYS

SEQ ID NO: 117 - protein, 108 aa, Artificial Sequence
  synthesized_CD47-31_VL
DIQMTQSPSSLSASVGDRVTITCRASQDVHSKVAWYQQKPGKAPKLLIYSTNRLYSGVPSRFSGSGSGTDFTLTISSLQPEDFATYYCQQHSNYPVTFGQGTKVEIKR

SEQ ID NO: 118 - protein, 10 aa, Artificial Sequence
  synthesized_CD47-31_CDR-H2
AINPFNGRTD

SEQ ID NO: 119 - protein, 116 aa, Artificial Sequence
  synthesized_CD47-31_VH
EVQLVESGGGLVQPGGSLRLSCAASGFTISNWWIHWVRQAPGKGLEWVAAINPFNGRTDYADSVKGRFTISADTSKNTAYLQMNSLRAEDTAVYYCTRGGYAMDYWGQGTLVTVSS

SEQ ID NO: 120 - protein, 7 aa, Artificial Sequence
  synthesized_CD47-32_CDR-L1
DVHYTVA

SEQ ID NO: 121 - protein, 9 aa, Artificial Sequence
  synthesized_CD47-32_CDR-L3
QQHFNFPIT

SEQ ID NO: 122 - protein, 108 aa, Artificial Sequence
  synthesized_CD47-32_VL
DIQMTQSPSSLSASVGDRVTITCRASQDVHYTVAWYQQKPGKAPKLLIYGTNRLYTGVPSRFSGSGSGTDFTLTISSLQPEDFATYYCQQHFNFPITFGQGTKVEIKR

SEQ ID NO: 123 - protein, 9 aa, Artificial Sequence
  synthesized_CD47-32_CDR-H1
FTINNFWIH

SEQ ID NO: 124 - protein, 10 aa, Artificial Sequence
  synthesized_CD47-32_CDR-H2
NISPFTGDTR

SEQ ID NO: 125 - protein, 116 aa, Artificial Sequence
  synthesized_CD47-32_VH
EVQLVESGGGLVQPGGSLRLSCAASGFTINNFWIHWVRQAPGKGLEWVANISPFTGDTRYADSVKGRFTISADTSKNTAYLQMNSLRAEDTAVYYCARGGYAMDIWGQGTLVTVSS

SEQ ID NO: 126 - protein, 106 aa, Artificial Sequence
  synthesized_CL sequence
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 330 aa, Artificial Sequence
  synthesized_CH sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 128 - protein, 6 aa, Artificial Sequence
  synthesized_linker-1
GGPGGP

SEQ ID NO: 129 - protein, 9 aa, Artificial Sequence
  synthesized_linker-2
GGPGGPGGP

SEQ ID NO: 130 - protein, 12 aa, Artificial Sequence
  synthesized_linker-3
GGPGGPGGPGGP

SEQ ID NO: 131 - protein, 15 aa, Artificial Sequence
  synthesized_linker-4
GGPGGPGGPGGPGGP

SEQ ID NO: 132 - protein, 5 aa, Artificial Sequence
  synthesized_linker-5
GGGGS

SEQ ID NO: 133 - protein, 10 aa, Artificial Sequence
  synthesized_linker-6
GGGGSGGGGS

SEQ ID NO: 134 - protein, 15 aa, Artificial Sequence
  synthesized_linker-7
GGGGSGGGGSGGGGS

SEQ ID NO: 135 - protein, 20 aa, Artificial Sequence
  synthesized_linker-8
GGGGSGGGGSGGGGSGGGGS

SEQ ID NO: 136 - protein, 25 aa, Artificial Sequence
  synthesized_linker-9
GGGGSGGGGSGGGGSGGGGSGGGGS